(19) United States
(12) Patent Application Publication
(10) Pub. No.: US 2024/0148315 A1
(43) Pub. Date: May 9, 2024

(54) XR-BASED PLATFORM FOR NEURO-COGNITIVE-MOTOR-AFFECTIVE ASSESSMENTS

(71) Applicant: SHEBA IMPACT LTD., Ramat Gan (IL)

(72) Inventors: Meir PLOTNIK-PELEG, Kfar Saba (IL); Yotam HAGUR-BAHAT, Tel Aviv (IL); Oran BEN-GAL, Ramat Hasharon (IL); Lotem KRIBUS-SHMIEL, Ramat Gan (IL); Evyatar ARAD, Modihin (IL); Meytal WILF, Givataim (IL); Noam GALOR, Tel Aviv (IL)

(73) Assignee: SHEBA IMPACT LTD., Ramat Gan (IL)

(21) Appl. No.: 18/281,374

(22) PCT Filed: Mar. 10, 2022
(86) PCT No.: PCT/IB2022/052172

§ 371 Date: Sep. 11, 2023

Related U.S. Application Data
(60) Provisional application No. 63/159,068, filed on Mar. 10, 2021.

(51) Int. Cl. A61B 5/00; A61B 5/11; G06F 3/01; G06T 19/00; G16H 50/30

(52) U.S. Cl. CPC A61B 5/4088; A61B 5/1124; A61B 5/1127; A61B 5/4884; A61B 5/7246; G06F 3/011; G06F 3/017; G06T 19/006; G16H 50/30

(57) ABSTRACT

A method of implementing an extended reality (XR) neuropsychological test of a person, including projecting onto an immersive display configured to display to the person three-dimensionally a series of virtual objects and conducting a cognitive test measuring executive functions using the virtual objects; simultaneously commanding sensors to monitor motor skills including at least one of: (i) gross hand movement by commanding a motion sensor, (ii) head movement by commanding a motion sensor, (iii) an eye movement of the person by commanding eye tracking, (iv) a gait by commanding at least one of mobility sensors, motion capture system sensors, force plate sensors or insole sensors and (v) postural stability by commanding at least one of mobility sensors, force plate sensors and insole sensors, and integrating the signals from the sensors so as to determine a neurocognitive and/or motor level of the person and produce output. Affect may also be measured.

### FIELD AND BACKGROUND OF THE INVENTION

The invention relates to extended reality neuropsychological tests that focus on multiple domains of the person tested. “Extended reality” (XR) refers to the various forms of computer-altered reality, including: Augmented Reality (AR), Mixed Reality (MR), and Virtual Reality (VR).

Neuropsychological tests of executive function have limited real-world predictive and functional relevance. The term “executive functions” is an umbrella term for a wide range of cognitive processes and behavioral competencies necessary for the cognitive control of behavior including problem solving, planning, sequencing, sustained attention, utilization of feedback, and multitasking. Neuropsychological tests of executive functions aim to assess these processes. Accordingly, performance on these tests is assumed indicative of executive functioning in everyday living. One of the limitations of these tests relates to their low “ecological validity”, namely the uncertainty about how closely they reflect capacity of executive function in real life. In this regard, Burgess et al. has claimed that the majority of neuropsychological assessments currently in use were developed to assess ‘cognitive constructs’ without regard for their ability to predict ‘functional behavior’.”

Neuropsychological assessment in virtual reality (VR).

Early discussions of ecological validity in neuropsychology emphasized that the technologies available at that time could not replicate the setting in which the behavior of interest actually occurs. Furthermore, currently, most neuropsychological assessments still use outdated methods (e.g., pencil-and-paper administration; static stimuli) that have yet to be validated with respect to real-world functioning. To overcome this limitation, testing participants in real word situations (e.g., the Multiple Errands Test [MET]) has been considered an ecologically valid and advantageous alternative to traditional tests. However, this approach is logistically challenging, requiring travel to a naturalistic testing sit. In an attempt to overcome this logistical hurdle, the Virtual Errands Test (VET) was devised by McGeorge et al. as an adaptation of the MET for VR-based administration. Still, this test, and similar VR variants, are limited in their ability to distinguish between healthy and clinical cohorts and to yield performance on the virtual tasks similar to performance in the real world.

Further, most VR-based tests like VET involve presenting a simulated VR environment on a standard computer screen (e.g., Elkind et al.), which may lead to a non-immersive experience, thus paradoxically compromising rather than enhancing ecological validity.

### SUMMARY OF THE EMBODIMENTS

A multimodal extended reality (XR) (in one implementation virtual reality (VR)) method/system synthesizes cognitive, motor, sensory and affect (e.g., emotion, arousal, motivation) domains in a manner that can only be done using extended reality, thereby providing a much deeper and an entirely different interaction with the person being tested. In one embodiment, there is a VR-based system/method that tests executive function in both sustained (Trails A) and divided attention (Trails B) of a color trail-making test in which the examinee needs to trace a sequence of numbered targets—one that utilizes a large-scale VR system (DOME-CTT) and the other that utilizes a portable head-mount display VR system (HMD-CTT).

The Color Trails Test (CTT) The Trail Making Test (TMT) are pencil-and-paper tests of executive function, attention and processing speed in research and clinical neuropsychological assessment. The Color Trails Test (CTT) is a culture-fair variant of the TMT. In Trails A the participant draws lines to sequentially connect circles numbered 1 to 25 (odd-numbered circles are pink; even-numbered circles are yellow). In Trials B the participant alternates between circles of two different colors (i.e., 1-pink, 2-yellow, 3-pink, 4-yellow, etc.). Scoring is based on the time needed to complete the tasks, with shorter time reflecting better performance. It has been proposed that Trails A assesses sustained visual attention involving perceptual tracking and simple sequencing, while Trails B more directly assesses executive function processes, including divided attention, simultaneous alternating and sequencing.

Applicant then evaluated construct validity, test-retest reliability, and age-related discriminant validity of the VR-based versions and explored effects on motor function. Methods: Healthy adults (n=147) in three age groups (young: n=50; middle-aged: n=80; older: n=17) participated. All participants were administered the original CTT, some completing the DOME-CTT (14 young, 29 middle-aged) and the rest completing the HMD-CTT. Primary outcomes were Trails A and B completion times (t_{A}, t_{B}). Spatiotemporal characteristics of upper-limb reaching movements during VR test performance were reconstructed from motion capture data. Statistics included correlations and repeated measures analysis of variance.

Results: Construct validity was substantiated by moderate correlations between the “gold standard” pencil-and-paper CTT and the VR method (DOME-CTT: t_{A} 0.58, 39 t_{B} 0.71; HMD-CTT: t_{A} 0.62, t_{B} 0.69). VR versions showed relatively high test-retest reliability (intraclass correlation; VR: t_{A} 0.60-0.75, t_{B} 0.59-0.89; original: t_{A} 0.75-0.85, 41 t_{B} 0.77-0.80) and discriminant validity (area under the curve; VR: t_{A} 0.70-0.92, t_{B} 0.71-0.92; original: t_{A} 0.73-0.95, t_{B} 0.77-0.95). VR completion times were longer than for the original pencil-and-paper test; completion times were longer with advanced age. Compared with Trails A, Trails B target-to-target VR hand trajectories were characterized by delayed, more erratic acceleration and deceleration, consistent with the greater executive function demands of divided vs. sustained attention; acceleration onset later for older participants.

Applicant has discovered that “ecological validity” is not merely related to the type of task performed and its relevance to daily living. In general, each response on a cognitive task involves interactions with sensory and motor functions, first to determine the required behavioral response and then to plan and execute it. These processes cannot be distinguished and examined with traditional pencil-and-paper testing or even with computerized testing platforms. Thus, as a first step, the VR neuropsychological test incorporate neuropsychological tests that measure particular cognitive constructs. These enhance ecological validity by including multi-multimodal (e.g., cognitive-sensory-motor) interactions, facilitating measurement of cognitive function in a manner more relevant to the interaction among multiple functions characteristic of everyday activities. Specifically, the VR technology employed allows for collection of quantitative three-dimensional kinematic data (unavailable for traditional neuropsychological tests) that tracks motion in space and may improve our ability to define and discriminate among levels of performance.

Applicant developed two VR systems/methods: (i) the DOME-CTT, designed for a large-scale VR system, in which the stimuli are projected on a 360° dome-shaped screen surrounding the participant, and (ii) the HMD-CTT, designed for a low-cost head-mount device (HMD), in which the stimuli are presented via VR goggles. In addition to developing the VR-based tests, Applicant evaluated their ability to measure the same cognitive constructs (construct validity) as the gold standard pencil-and-paper CTT, as well as their ability to differentiate among healthy young, middle-aged and older age groups (discriminant validity) relative to the original CTT. Then Applicant explored cognitive-motor interactions during performance of the VR-CTT tasks. Finally, Applicant developed a dramatically new cognitive-motor-affect assessment method/system in Extended Reality that introduces new interactions and uses new methods.

One embodiment is a computer-implemented method of implementing an extended reality (XR) neuropsychological test of a person using software stored on a non-transitory computer-readable medium, the software executed by a processor, the software performing the following: projecting onto an immersive display, that is configured to display to the person three-dimensionally, a series of virtual objects and conducting a first cognitive test that measures cognitive skills, including executive functions, using the series of virtual objects; while measuring the cognitive skills involving the executive functions, commanding sensors to monitor motor skills of the person including at least one of: (i) gross hand movement by commanding a motion sensor, (ii) head movement by commanding a motion sensor, (iii) an eye movement at the virtual objects by commanding an eye tracking sensor, (iv) a gait of the person by commanding at least one of mobility sensors, motion capture system sensors, force plate sensors or insole sensors and (v) postural stability of the person by commanding at least one of mobility sensors, force plate sensors and insole sensors, and integrating the signals from the sensors so as to determine a neurocognitive level or a motor skills level of the person and produce an output reflecting the determined level.

In some embodiments, the monitoring of the motor skills includes the eye tracking and monitoring the head movement,

In some embodiments, the monitoring of the motor skills includes monitoring the head movement, the gross hand movement and the gait of the person.

In some embodiments, the monitoring of the motor skills includes monitoring the gait of the person while the person is doing at least one of walking, running and standing.

In some embodiments, the method further comprises applying to the person movement related perturbations to generate physical stress or increased difficulty during the monitoring of the motor skills and/or the testing of the cognitive skills.

In some embodiments, the method further comprises at least one of (a) sensing an affect of the person by sensing at least one of galvanic skin response (GSR), ECG, respiration and skin temperature; and (b) applying sensory signals in an XR environment including by using at least one of (i) colors or shapes, (ii) sounds, and (iii) tactile sensations to affect an outcome of the cognitive testing, motor skills monitoring and/or affect sensing of the person.

In some embodiments, the executive functions include at least one of planning, sustained visual attention (SVA), divided attention (DA), spatial orientation and task switching, sequencing or a correlation between any two executive functions.

In some embodiments, the executive functions include at least two of planning, sustained visual attention (SVA), divided attention (DA), episodic memory, spatial orientation and task switching, sequencing, mental flexibility, visual scanning, information processing, problem solving, abstraction, impulse control.

In some embodiments, the method further comprises sensing motor skill kinematics of the person and dividing, by the processor, a motor skill kinematic waveform into at least one planning portion and at least one execution portion while monitoring a velocity of a body portion of the person to ascertain a planning period ratio that correlates with the person's processing speed and motor execution speed. In some embodiments, the method further comprises extracting a level of hand movements during the planning periods to obtain indications of a level of impulsivity versus caution of the person.

In some embodiments, the gross hand movement involves reaching movements by the person to hit the virtual objects.

In some embodiments, the eye tracking and a pupil dilation are recorded to assess cognitive processing and visual exploration patterns, and wherein an invisible unity object representing a gaze position at any given moment records three-dimensional coordinates of the gaze along with three-dimensional coordinates of the hand and head of the person.

In some embodiments, the method further comprises comparing a waveform of the gross hand movement and a waveform of an eye movement of the person.

In some embodiments, the method further comprises comparing a waveform of the gross hand movement and a waveform of the head movement of the person to measure head-hand coordination.

In some embodiments, the method further comprises the software performing operating a moving platform or treadmill configured for the person to stand, walk or run on so as to introduce physical perturbations. In some embodiments, the motion capture system sensors are reflective markers situated on a body of the person and wherein the motor skills are monitored while the person is walking on the treadmill using self-paced walking, fixed paced walking, or walking overground while immersed in the virtual environment or extended reality.

In some embodiments, the method further comprises determining, by the processor, the neurocognitive and motor level of the person based at least in part on at least one of: (a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching; (b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching; (c) changing a level of physical difficulty of motor skills or cognitive skills by applying or changing physical perturbations in response to detection of a particular affective state; (d) comparing the motor skills of the person utilizing only eye movement by the person, with motor skills of the person using only head movement by the person, or comparing motor skills using one hand of the person only versus motor skills using both hands of the person; and (e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

In some embodiments, the method further comprises determining, by the processor, the neurocognitive and motor level of the person based at least in part on at least two of: (a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching; (b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching; (c) changing a level of physical difficulty of motor skills by applying or changing physical perturbations in response to detection of a particular affective state; (d) comparing the motor skills of the person utilizing only eye movement by the person, with motor skills of the person using only head movement by the person, or comparing motor skills using one hand of the person only versus motor skills using both hands of the person; and (e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

In some embodiments, the method further comprises generating a multiplicity of configurations of the extended reality neuropsychological tests of the person by: projecting onto the immersive display to the person three-dimensionally during the first cognitive test and during a second cognitive test in a first configuration, the second cognitive test configured to display the series of virtual objects so as to distract an attention of the person more than the first cognitive test; wherein a number, axis, angle and order of rotations and spatial flips are randomized, rotating a scene viewable on the display screen by the person along at least one of the X, Y and Z axes and spatially flipping the scene, to create a new configuration such that (i) a total distance of a trail from the first virtual object to the last virtual object of a particular cognitive test is unchanged from one configuration to another configuration and such that (ii) the distance and trajectory between any one of the virtual objects and a next numbered virtual object are identical between the first cognitive test of a particular configuration and the second cognitive test of the particular configuration. In some embodiments, the method further comprises generating thousands of configurations of the extended reality neuropsychological tests of the person by varying an angle of the rotations.

In some embodiments, the method further comprises various scoring a performance of the person on the neurocognitive level based on a completion time of the test or based on a single or a range of numeric or alphabetic indicia.

In some embodiments, the method further comprises determining a score for the output including by contrasting the person's performance under a first condition with the person's performance under a second condition that differs from the first condition with respect to one or more test parameters.

In some embodiments, the virtual objects are perceived by the person as moving in different directions and/or at different rates.

Another embodiment is a method of implementing extended reality (XR) neuropsychological tests of a person using software stored on a non-transitory computer-readable medium, the software executed by a processor, the software performing the following: projecting onto an immersive XR display, that is configured to display to the person three-dimensionally, a series of sequentially identified virtual targets so as to conduct a first cognitive test that measures executive functions and so as to conduct a second cognitive test that measures executive functions, the second cognitive test configured to display the series of virtual targets so as to distract an attention of the person more than the first cognitive test; receiving input from at least one of (i) a handheld and (ii) head-mounted input device that is moved by the person from one virtual target to another until a predefined number of the virtual targets have been contacted in a predefined order, and instructing the display to update the XR display so as to record visual connections between the targets that track a movement of the handheld input device, when used, and of the head-mounted input device, when used; commanding sensors, during each of the first cognitive test and the second cognitive test to monitor motor skills of the person including at least two of (i) gross hand movement by commanding a motion sensor, (ii) head movement by commanding a motion sensor, (iii) an eye movement of the person at the virtual targets by commanding an eye tracking sensor, (iv) a gait of the person by commanding at least one of mobility sensors, motion capture system sensors, force plate sensors or insole sensors and (v) postural stability of the person by commanding at least one of mobility sensors, force plate sensors and insole sensors; and integrating the signals from the sensors so as to determine a neurocognitive level or a motor skills level of the person and produce and output reflecting the determined level.

In some embodiments, the monitoring of the motor skills includes the eye tracking and monitoring the head movement.

In some embodiments, the monitoring of the motor skills includes monitoring the head movement, the gross hand movement and the gait of the person.

In some embodiments, the monitoring of the motor skills includes monitoring the gait of the person while the person is doing at least one of walking, running and standing.

In some embodiments, the method further comprises applying to the person movement related perturbations to generate physical stress or increased difficulty of performance during the monitoring of the motor skills and/or the testing of the cognitive skills.

In some embodiments, the method further comprises at least one of (a) sensing an affect of the person by sensing at least one of galvanic skin response (GSR), ECG, respiration and skin temperature; and (b) applying sensory signals in an XR environment in order to manipulate mental stress levels, including by using at least one of (i) colors or shapes, (ii) sounds, (iii) tactile sensations to affect an outcome of the cognitive testing, motor skills monitoring and/or affect sensing of the person.

In some embodiments, the executive functions include either sustained visual attention (SVA) or divided attention (DA) and at least one of planning, spatial orientation and task switching, sequencing or a correlation between any two executive functions.

In some embodiments, the executive functions include either sustained visual attention (SVA) or divided attention (DA) and at least two of planning, episodic memory, spatial orientation and task switching, sequencing, mental flexibility, visual scanning, information processing, problem solving, abstraction, impulse control.

In some embodiments, the method further comprises sensing hand kinematics of the person and dividing, by the processor, a hand kinematic waveform into at least one planning portion and at least one execution portion while monitoring a velocity of a hand of the person to ascertain a planning period ratio that correlates with the person's processing speed and motor execution speed.

In some embodiments, the method further comprises extracting a level of hand movements during the planning periods to obtain indications of a level of impulsivity versus caution of the person.

In some embodiments, the gross hand movement involves reaching movements by the person to hit the virtual targets.

In some embodiments, the eye tracking and a pupil dilation are recorded to assess cognitive processing and visual exploration patterns, and wherein an invisible unity object representing a gaze position at any given moment records three-dimensional coordinates of the gaze along with three-dimensional coordinates of the hand and head of the person.

In some embodiments, the method further comprises comparing a waveform of the gross hand movement and a waveform of an eye movement of the person.

In some embodiments, the method further comprises comparing a waveform of the gross hand movement and a waveform of the head movement of the person to measure head-hand coordination.

In some embodiments, the method further comprises the software performing operating a moving platform or treadmill configured for the person to stand, walk or run on so as to introduce physical perturbations. In some embodiments, the motion capture system sensors are reflective markers situated on each foot of the person and wherein the motor skills are monitored while the person is walking on the treadmill using self-paced walking, fixed paced walking, or walking overground while immersed in the virtual environment.

In some embodiments, the method further comprises determining, by the processor, the neurocognitive and motor level of the person based at least in part on at least one of: (a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching; (b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching; (c) changing a level of difficulty of performance of motor skills by introducing physical perturbations in response to detection of a particular affective state; (d) whether the motor skills were monitored utilizing eye movement by the person only, head movement by the person only, one hand of the person only or both hands of the person only; and (e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

In some embodiments, the method further comprises determining, by the processor, the neurocognitive and motor level of the person based at least in part on at least two of: (a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching; (b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching; (c) changing a level of performance of motor skills by introducing physical perturbations in response to detection of a particular affective state; (d) whether the motor skills were monitored utilizing eye movement by the person only, head movement by the person only, one hand of the person only or both hands of the person only; and (e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

In some embodiments, the method further comprises generating thousands of spatially distinct configurations of the extended reality neuropsychological tests of the person by: projecting onto the immersive display to the person three-dimensionally during the first cognitive test and during a second cognitive test in a first configuration, the second cognitive test configured to display the series of virtual targets so as to distract an attention of the person more than the first cognitive test; wherein a number, axis, angle and order of rotations and spatial flips are randomized, rotating a scene viewable on the display screen by the person along at least one of the X, Y and Z axes and spatially flipping the scene, to create a new configuration such that (i) a total distance of a trail from the first virtual target to the last virtual target of a particular cognitive test is unchanged from one configuration to another configuration and such that (ii) the distance and trajectory between any one of the virtual targets and a next numbered virtual target are identical between the first cognitive test of a particular configuration and the second cognitive test of the particular configuration.

In some embodiments, the method further comprises generating an unlimited number of configurations of the extended reality neuropsychological tests of the person by varying an angle of the rotations.

In some embodiments, the method further comprises scoring a performance of the person on the neurocognitive level based on a completion time of the test or based on a single or range of a numeric or alphabetic scores.

In some embodiments, the virtual targets are dynamic in that they are perceived by the person as moving in different directions and/or at different rates.

In some embodiments, the method further comprises determining a score for the output including by contrasting the person's performance under a first condition with the person's performance under a second condition that differs from the first condition with respect to one or more test parameters.

### BRIEF DESCRIPTION OF THE DRAWINGS

Various embodiments are herein described, by way of example only, with reference to the accompanying drawings, wherein:

FIG. 1 is a schematic illustration of the interaction of nodes representing particular competence parameters measured or sensory effects supplied within various domains as well as a correlation matrix in accordance with one embodiment;

FIG. 2 is a schematic illustration as in FIG. 1 and also showing analog signals of motor and affect domains in accordance with one embodiment;

FIGS. 3A-3B show waveforms of head and hand motor skill measurements and cross-correlation between them on the right it shows two graphs including a cross-correlation graph and a LAG graph as used in accordance with one embodiment;

FIG. 4 shows a correlation matrix in accordance with one embodiment;

FIG. 5 is an illustration of the creation of different configurations of the scenes viewable by a person in the ER environment in accordance with one embodiment;

FIG. 6 is a schematic illustration of a cognitive-motor-affective interaction paradigm in accordance with one embodiment,

FIGS. 7A-7B show motor skills test results involving hand kinematics divided between planning and execution in accordance with one embodiment;

FIGS. 8A-8C illustrate examples of three different visual scenery cues of different stress-inducing level used in modulating affect in accordance with one embodiment;

FIG. 9 is an illustration of a cognitive skills test called the London Bridge Test;

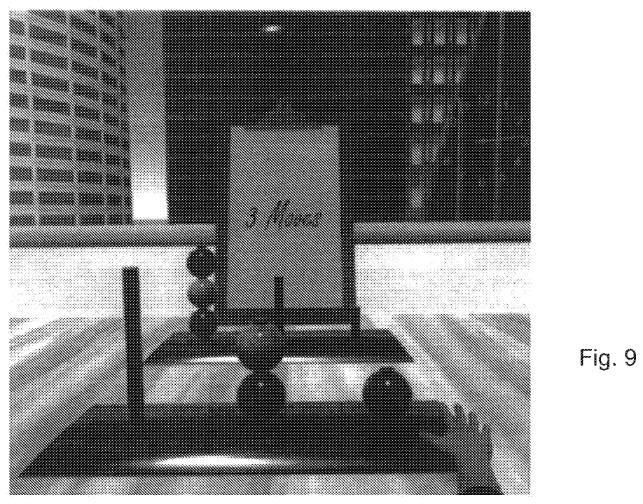

FIG. 10 is a flow chart showing a method in accordance with one embodiment;

FIG. 11 is a flow chart showing a further method in accordance with one embodiment; and

FIG. 12 is a schematic of a computer system that includes software for implementing methods in accordance with certain embodiments.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention, since the scope of the invention is best defined by the appended claims.

The invention generally provides a method and system for making a neuro-cognitive-motor-affective determination, including for example a score, for assessments (whether in-clinic or via tele-rehabilitation or any assessment/evaluation setting) in relation to diagnosis, monitoring, treatment and/or rehabilitation and may be used for people who do or may suffer from stroke, Parkinson's, neurodegenerative diseases, musculoskeletal diseases and/or bruises. The method/system may also be useful for assessing the neuro-cognitive-motor-affective performance of high performance people such as sportsmen/women and rescue workers, and also for evaluating any candidate for job positions. The score may be a cognitive, motor or cognitive-motor score. The score may be computed based on kinematic analysis of the measured motor skills. The score may be numeric, alphabetical, continuous, non-continuous, an amount of time to complete a portion or all of the test, a level of difficulty or any other appropriate score. The score may stand by itself or may be determined relative to a baseline of that individual or a baseline of the population or of a portion of the population with similar characteristics to that individual. The baseline data may already be available.

The term “neurocognitive” and cognitive” are used herein interchangeably. “Neurocognitive” tends to be used more when discussing a determination of a score or level of the person in that area.

The following represent non-limiting examples of how the Extended-Reality CAMA (cognitive-affective-motion interaction assessment) method and system may be applied.

The first example of the use of the CAMA method or system is clinical diagnosis: assessment of cognitive-motor-affective-sensory capabilities of person after cerebral vascular accident (CVA). The applicable population are those who are after a cerebral vascular accident (CVA). The goal is assessment of the cognitive-motor-affective-sensory capabilities of these individuals. In one embodiment, a cognitive test is set up. In one particular non-limiting implementation the cognitive test can be an XR-CTT form A and form B (sustained visual attention, divided attention respectively), and/or e.g., the XR tower of London (planning, spatial orientation) and/or e.g., the XR block design test (non verbal abstract conceptualization, spatial visualization and motor skills), and/or e.g., the XR corsi block test (visuo-spatial short-term working memory) and the person will perform using, e.g., head mount device while, e.g., standing and/or sitting, and/or walking. The measurements taken will be motor measurements including for example eye tracking, head rotations, gross manual movements, grasping movements, posture and gait analysis. These will be recorded from position and kinematic data.

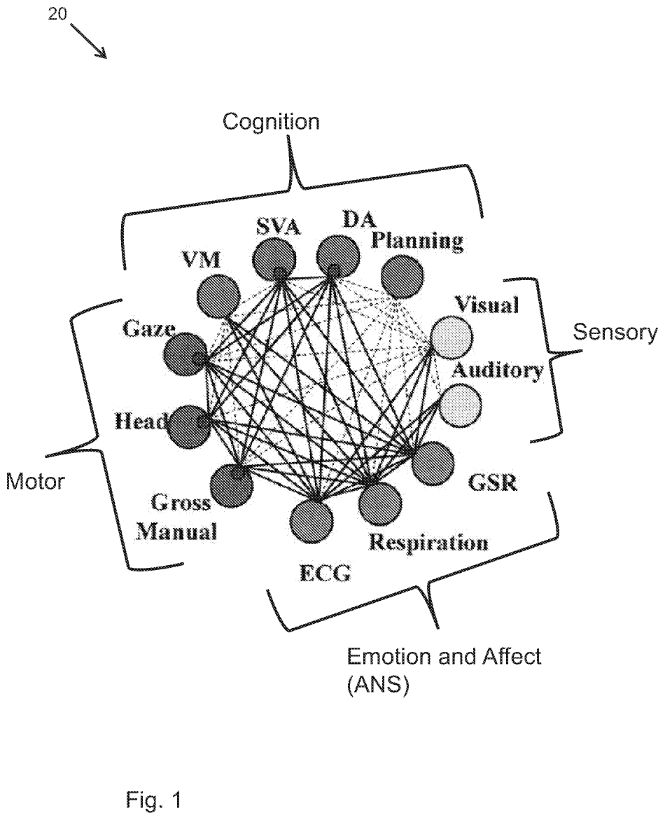

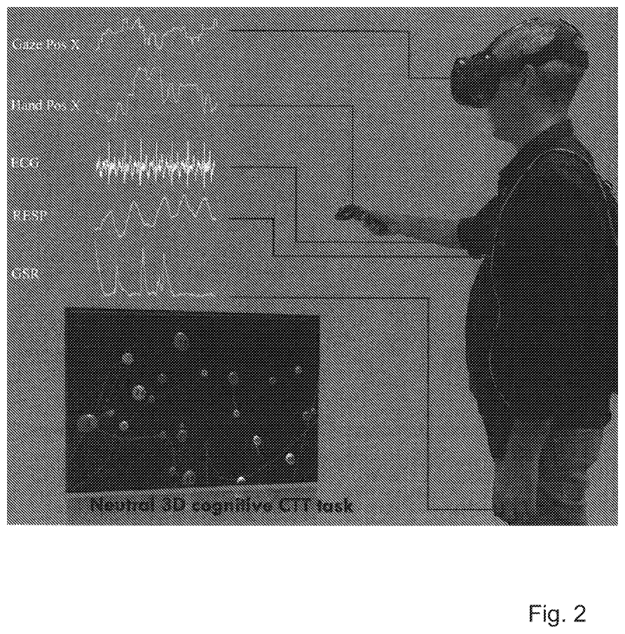

Additional measurements may include autonomic nervous system (“ANS”) monitoring indicative of affective state such as electrocardiogram (ECG) and/or galvanic skin response (GSR) and/or respiration (FIG. 1, FIG. 2). Electroencephalography (EEG) and/or fNIRs will be recorded to monitor cerebral activation.

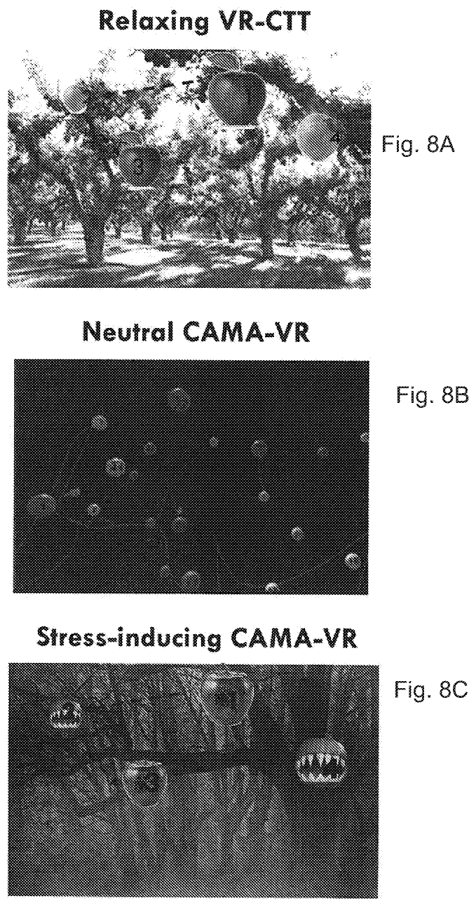

The performance of the individual, in each domain namely cognitive, motor and affect will be recorded under a variety of sensory conditions, e.g., a variety of visual conditions (darker/lighter VR environments, types of visual environmental scenery that induce stress or relaxation as shown in FIGS. 8A-8C), and/or a variety of auditory conditions (volume of background sounds, and types of sounds and/or a variety of tactile sensations such as using a haptic glove to produce vibrations over the palm of the hand so as to determine the effect of mental stress and physical stress on the individual's performance.

For example, if a person's head rotation and the person's hand movement are both tracked while monitoring a cognitive skill, e.g., sustained visual attention (SVA), and the ratio (which is a type of “correlation coefficient”) between them is 0.9 (the head and the hand move in a highly spatially-coherent manner, but not in perfect unison, e.g., for every 100 hand movements there are 90 head movements). If the same motor skills (head rotation and hand movement) are then similarly tracked while monitoring a different cognitive skill, e.g., divided attention (DA) which adds cognitive stress, the ratio between them is for example 0.778 (for every 90 hand movements there are 70 head movements). Then it can also be said that for hand movement (a type of motor skill) alone, the effect of DA compared to SVA is 0.9 (90/100) and for head movement (a type of motor skill) alone, the effect of DA compared to SVA is about 0.77 (70/90). Furthermore, it can be also stated that for the ratio between hand movements and head movements, the effect of DA compared to SVA is about 0.77/0.9 or about 0.86.

Instead of head and hand motor skills one can use eye and hand motor skills or any other motor skills. In addition, instead of DA compared to SVA any other cognitive skills can be used.

In the context of motor skills performance, this formula may be generalized as motor skill performance X and Y under conditions of a cognitive skill test A or cognitive skill test B (or X_{A} and YA compared to X_{B} and Y_{B}).

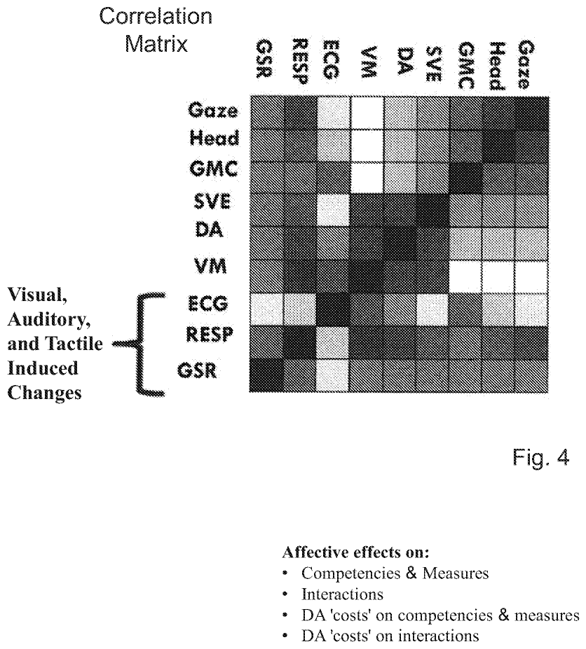

As shown by FIG. 4, a similar performance assessment outcome can be done with other motor skills and any other cognitive skills as well as with any affect domain tests of the individual. The gradations of shading represent degrees of correlation in the correlation matrix. One non-limiting example of a formula relating to costs is: Cost of stress (on head hand coordination)=[(phase during stress)−(phase no stress)]/(phase no stress).

FIG. 1 is a schematic illustration of the interaction of nodes representing particular competence parameters measured or sensory effects supplied within various domains. It shows interactions of cognitive, motor, affective and sensory domains wherein the small circles are examples of specific competencies within each domain. In general, looking at the three testing domains of cognitive, motor and affect (as seen in FIG. 1) this can be done for any of the domains (cognitive-motor-affect) while changing the cognitive, motor, affective or sensory conditions. Furthermore, test outcomes on the examined performance may be assessed with respect to the interactions between the performance in any of the nodes in FIG. 1 in view of changes in any of the other nodes.

All features may be compared to baseline features (of the individual or of the population) extracted while focusing on motor, or cognitive, or affective performance alone or in any combinations, under different controlled sensory conditions (e.g., performing the cognitive task using only the eyes with no hand movements, removing the cognitive task and performing only the hand movements, etc.).

In a second illustrative use of CAMA, the features of the tests may be changed to accommodate for inter-individual variability in affective states and traits. For example, one may compare a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching. Another example is changing a level of difficulty of performance of the motor skill by inducing physical perturbation in response to detection of a particular affective state. For example, if the person has anxiety and it is not necessary to test for anxiety (for example because the person has a desk job), then one may reduce the stress perturbation level for example by altering the sensory signals to create a more relaxing virtual environments or changing the cognitive testing, e.g., by presenting an easier task with less cognitive load. The physical perturbation can for example be an unexpected movement of a treadmill or platform or floor that the person is standing or walking or running on. A further example is vibrations induced by a haptic glove.

Baseline recordings of ANS (emotion, arousal, motivation (affect) signals (as described in use case one) will be taken in neutral environments (e.g., XR environment of white room). Tests (as described in the first example of the use of the method/system) will be employed. If ANS signals indicate elevation of stress, then tests features, e.g., spatial disperse of targets balls in the CTT will be modified.

A third illustration of the use of the CAMA XR assessment method or system involves performance screening in a job interview. The population: A candidate for a type of work that requires effortful thinking and motor functioning under stressful conditions (e.g., personal assistant of a high-tech manager). The goal: assess how the individual's cognitive abilities are affected by stress level. The setup: Applying a combination of cognitive and motor skills performance tests (one non-limiting example is CTT A+B) under two modes of action—with a neutral virtual environment, and the same tasks (with different spatial layout) with a stress-inducing virtual environment.

In this case, the measurements may be the time it takes to complete the test (completion times), head-hand coordination, visual search efficiency with number of scanned targets (using eye tracking) or objects. Test outcome: the ‘cost’ (i.e., the reduction in the person's performance on the test) stemming from having induced environmental stress on each of the measurements. Candidates will be scored according to the amount that interference stress induces on their cognitive-motor performance.

A fourth example of the use of the XR CAMA method/system involves the diagnosis of a medical intern's fatigue. Using, for example the CTT test with ‘mirror-reversal’ element (which introduces an element of the more difficult task of motor learning), every medical intern will take the test at the beginning of a shift and after every 8 hours. The first test will be used as the base line of the intern performance A decline of specific percentage in performance will indicate that the intern is in no condition to make life-effecting decisions. In this case, the intern will be obliged to get a rest. After the rest, another test will be taken and its performance will determine if the intern can get back to the shift. This application may also be useful and valid for truck and bus drivers, heavy machinery operators, pilots and any other high-risk professions. The principles and operation of an XR-Based Platform for Neuro-Cognitive-Motor-Affective Assessments may be better understood with reference to the drawings and the accompanying description.

Applicant initially demonstrated the feasibility and validity of a virtual reality cognitive test. Two VR-CTT platforms were developed: DOME-CTT and HMD-CTT. Findings from experiments using these platforms are described in Study 1 and Study 2, respectively. There were a total of 147 healthy participants in Study 1 and Study 2 who completed this testing as part of larger experimental protocols. Participants were subdivided into the following age groups: (1) young adults (YA), ages 18-39 years (n=50); (2) middle-aged adults (MA) ages 40-65 years (n=80); and (3) older adults (OLD), ages 65-90 years (n=17). For all groups, exclusion criteria were motor, balance, psychiatric or cognitive conditions that may interfere with understanding the instructions or completing the required tasks (determined by screening interviews). The protocols were approved by the Sheba Medical Center institutional review board (IRB), and all participants signed informed consent prior to enrolling in the study. Methods for Study 1 (DOME-CTT) Participants: Data from 14 YA (age: 27.9±5.0 [mean±SD] years, education: 16.4±2.9 [mean±SD] years; 9 females) and 29 MA (age: 55.8±6.2 years, education: 16.3±3.0 155 years; 16 females) were included in Study 1.

### Apparatus

A fully immersive virtual reality system (CAREN High End, Motek Medical, The Netherlands) projected a virtual environment consisting of the task stimuli on a full-room dome-shaped screen surrounding the participant. The system comprises a platform with an embedded treadmill and is synchronized to a motion capture system (Vicon, Oxford, UK). Auditory stimuli and feedback are delivered via a surround sound system.

### A Color Trails Test was Developed for Large-Scale VR—the DOME-CTT

A virtual version of the CTT was developed to demonstrate the feasibility of performing neuropsychological testing in a virtual environment. The original pencil-and-paper CTT consists of four parts: practice (Trails) A, test (Trails) A, practice (Trails) B and test (Trails) B. In the VR version of the CTT, the two-dimensional (2D) page is replaced with a three-dimensional (3D) VR space that introduces the dimension of depth to the target balls (that replace the 2D circles) and to the generated trajectory and the following rules were followed: (1) balls were positioned so that virtual trajectories between sequential target balls would not cross previous trajectories (i.e., between target balls from earlier in the task sequence); (2) proximity of balls in a given region of the 3D space was similar to that in the corresponding region of 2D space in the original CTT; (3) for Trails B, Applicant positioned the corresponding identically-numbered distracter ball of incorrect color at a relative distance to the target ball similar to the that in the original 2D CTT.

The participant performed the DOME-CTT with a marker affixed to the tip of a wand-like pointing stick held in the dominant hand (corresponding to the pen or pencil in the original CTT). The three-dimensional coordinates of the marker were tracked in real time by the motion capture system at a sampling rate of 120 Hz. A virtual representation of this marker appeared within the visual scene (i.e., ‘avatar’, represented by a small red ball). To mimic drawing lines in the 2D pencil-and-paper CTT, as the participant moved his/her hand within the VR space, a thick red ‘tail’ trailed directly behind the position of the (red ball) avatar, gradually becoming a faint yellow tail as the avatar moved farther away from the initial position.

Movement of the marker was recorded in real time by a motion capture system that allows the reconstruction of kinematic data over the duration of the test. In addition to the apparatus, the testing procedure was also adapted for the new format. As above, the original pencil-and-paper CTT comprises four consecutively administered test levels: (1) Trails A practice; (2) Trails A; (3) Trails B practice; and (4) Trails B. Though drawing lines with a pen/pencil on a piece of paper is highly familiar, manipulation of the VR ‘controller’ (i.e., the marker affixed to the pointing stick) to move an avatar (i.e., the red ball) within the virtual environment is a relatively unfamiliar skill. Thus, the DOME-CTT began with an additional practice level in which participants practiced guided movement of the avatar within the virtual space to so that it touched the numbered ball targets. During this level, participants were introduced to the positive feedback received when the avatar ball touched the correct ball (i.e., 10 momentary enlargement of the ball) and the negative feedback when it touched an incorrect ball (i.e., brief buzzing sound). These feedback stimuli were also presented during the remainder of the testing session. After this initial practice level, test levels corresponding to those in the original CTT were administered. However, unlike the pencil-and-paper CTT, Trails A and Trails B were each preceded by two different practice levels. In the first practice level, all virtual balls were clustered near the center of the visual field, and in the second practice level, the balls were distributed throughout the visual field, approximating the spatial distribution of the balls in the actual testing levels.

### Procedure

Data on pencil-and-paper CTT and DOME-CTT were collected as part of three different experimental protocols. All data (with the exception of test retest data) described in this study were collected on the first visit. The participants completed the pencil-and-paper CTT and DOME-CTT on the same day in counterbalanced order across participants. Applicant monitored the general wellbeing of the participants (e.g., absence of fatigue) throughout the tests. Outcome measures and statistical analysis For the pencil-and-paper CTT and the DOME-CTT, completion times for Trails A and B were recorded (tA, tB, respectively). Construct validity was assessed by correlating tA and tB from the DOME-CTT with the corresponding scores from the gold standard CTT (Pearson coefficient). Analysis of variance (ANOVA) was used to assess effects of Group (young, middle aged; between-subjects factor), Trails (Trails A, Trails B; within-subjects factor) and Format (pencil-and-paper CTT, DOME-CTT; within-subjects factor). Partial Eta Squared was computed as a measure of effect size. To verify suitability of parametric statistics, Shapiro-Wilk normality tests were run for each outcome variable per group. Of the eight normality tests, none indicated non-normal distributions (Shapiro-Wilk statistic ≤0.93; p≥0.16). Levene's test revealed inhomogeneity of variance among groups for Trails A and B in pencil-and-paper and VR formats (p0.05). Descriptive statistics, figures and correlations analyses were performed on the pre transformed data.

Summary statistics (mean±SD) were computed for t_{A} and t_{B} from the pencil-and-paper CTT and DOME-CTT.

Errors were manually recorded by the experimenter for the pencil-and-paper CTT; for the DOME-CTT, errors were recorded both manually and automatically by the software. Related samples Wilcoxon Sign Test (non-parametric) test was used to evaluate the Format effect separately for Trails A and B. Mann-Whitney U tests were used to evaluate the group effect.

To examine discriminant validity (i.e., ability to separate between YA and MA) of the DOME-CTT as compared with the pencil-and-paper CTT, Applicant plotted receiver operating characteristic curves (ROC) for Trails A and Trails B (i.e., tA and tB, respectively) for each test format and calculated the area under the curve (AUC; range: 0-1, higher values reflect better discriminability).

Level of statistical significance was set at 0.05. Statistical analyses were run using SPSS software (SPSS Ver. 24, IBM).

### Methods for Study 2

### Participants

Data from 36 YA (age: 26.7±4.1 [mean±SD] years, education: 15.9±2.3 [mean±SD]; 21 females), 51 MA (age: 56.2±6.2 years, education: 16.8±3.0 years; 39 females) and 17 OLD (age: 73.7±6.5 years, education: 13.1±2.7 years; 11 females) were included in Study 2.

Apparatus VR technologies have advanced rapidly in recent years. In addition to new technical features for precise stimulus delivery and response measurement, as well as enhanced usability, low-cost VR is now widely accessible. The most accessible type of VR system is the head-mount device (e.g., HTC Vive, Oculus Rift), which is designed for home-based operation. In addition to its continued popularity for entertainment, VR is now being applied in a variety of ‘serious’ contexts, ranging from surgical simulation to the study of human performance and psychological function (30, 31). For this study, Applicant used a fully immersive VR system (HTC-Vive; New Taipei City, Taiwan) including a headset with ˜100c field of view (FOV) in the horizontal plane and ˜110c FOV in the vertical plan. Also included were a controller for user interaction with the virtual environment and two ‘lighthouse’ motion trackers for synchronizing between actual controller position and corresponding position in the virtual environment. The headset-based VR system called the HMD-CTT

In developing the HMD-CTT version, Applicant adopted a similar approach to the development of the DOME-CTT. With the exception that the participant held the HTC controller rather than a wand-like pointing stick, task design matched the DOME-CTT, including positive and negative feedback cues, practice and 13 test procedures. The HMD-CTT incorporated practice and test levels corresponding to those in the DOME-CTT described above.

### Procedure

The procedure was identical to that of Study 1 (see above).

### Outcome Measures and Statistical Analyses

For the pencil-and-paper CTT as well as the HMD-CTT, completion times for Trails A and B were recorded (tA, tB, respectively). Similar to Study 1, construct validity of tA and tB was assessed by correlating tA and tB from the HMD-CTT with the corresponding scores from the gold standard pencil-and-paper CTT. As in the DOME-CTT study, repeated-measures ANOVA was used to assess the effects of Group (YA, MA, OLD; between-subjects factor), Trails (Trails A, Trails B; within-subjects factor) and Format (pencil-and-paper CTT, HMD-CTT; within-subjects factor). Partial Eta Squared was computed as a measure of effect size. Applicant used the Bonferroni correction to adjust for multiple comparisons in the post-hoc pairwise comparisons.

As for Study 1, Shapiro-Wilk normality tests were run for each outcome variable per group to verify the suitability of parametric statistics. Of the twelve normality tests, four indicated non-normal distributions: tA pencil-and-paper CTT, MA group, Shapiro-Wilk statistic=0.786, p=0.002; tB pencil-and-paper CTT, MA group, Shapiro-Wilk statistic=0.770, p=0.002; tB pencil-and-paper CTT, YA group, Shapiro-Wilk statistic=0.744, p=0.001, tA pencil-and-paper CTT, OLD group, Shapiro-Wilk statistic=0.844, p=0.015. As in Study 1, Levene's test. revealed inhomogeneity of variance among groups for Trails A and B in pencil-and-paper and VR formats (p<0.05). Descriptive statistics, figures and correlations analyses were performed on the pre transformed data. Additional analyses are described in the Results section.

Regarding prevalence of errors, related samples Wilcoxon Sign Test (non-parametric) test was used to evaluate the Format effect separately for Trails A and B. Kruskal-Wallis tests were used to evaluate the group effect (three levels) separately for Trails and Format.

As for Study 1, level of statistical significance was 0.05, and statistical analyses were run with SPSS.

### Qualitative Analysis of Manual Performance

Spatial coordinates of the controller position (corresponding to ‘red ball’ avatar) were recorded throughout the HMD-CTT sessions. Custom software written in MATLAB (Mathworks, Inc.) used this data to extract and analyze the 24 target-to-target reaching movements during Trails A and Trails B, respectively (errors were excluded from this analysis). Applicant made a qualitative assessment of the trajectories generated in each of the three groups by examining the grand averages of their velocity profiles to characterize upper-limb motor behavior associated with the HMD-CTT tasks. For a full description of the methodology used to generate these grand averages.

### Evaluation of Test-Retest Reliability (Study 1 and Study 2)

To evaluate test-retest reliability, some participants completed a second assessment.

Fifteen MA participants from Study 1 completed an additional evaluation about 12 weeks after the initial evaluation during which they completed the pencil-and-paper CTT and DOME-331 CTT in the same order as in the initial evaluation.

Thirty-two MA participants from Study 2 completed an additional evaluation about 12 weeks after the initial evaluation. Also from Study 2, twenty participants (n=10 334 YA, n=1 MA, n=9 OLD) completed an additional evaluation 2 weeks after the initial one. The pencil-and-paper CTT and HMD-CTT were administered in the same order as in the initial evaluation. To assess test-retest reliability, Applicant computed intraclass correlation coefficients (ICC; two-way mixed, effects, absolute agreement) for tA and tB scores from the traditional pencil-and-paper CTT and the DOME-CTT (Study 1) or HMD-CTT (Study 2) collected at two visits. ICC reflects similarity of the obtained scores irrespective of the level of performance, reflecting not only correlation, but also agreement between measurements. By convention ICC >0.75 is considered good reliability.

### RESULTS Study 1

### Performance on the DOME-CTT: Group, Trails and Format Effects

All participants completed the tests in both formats. Time for initial DOME-CTT practice levels varied between participants, but usually did not exceed 10-15 min. Due to technical malfunction, data for DOME-CTT Trails A from one YA participant was not recorded.

Statistical analysis revealed effects of Group (F1,40=25.4, p.30). As participants in the OLD group had significantly fewer years of education than participants in the YA and MA groups (p #0.001; see Methods), Applicant repeated the analysis entering years of education as a covariate. The results did not change appreciably (see supplementary material file #1).

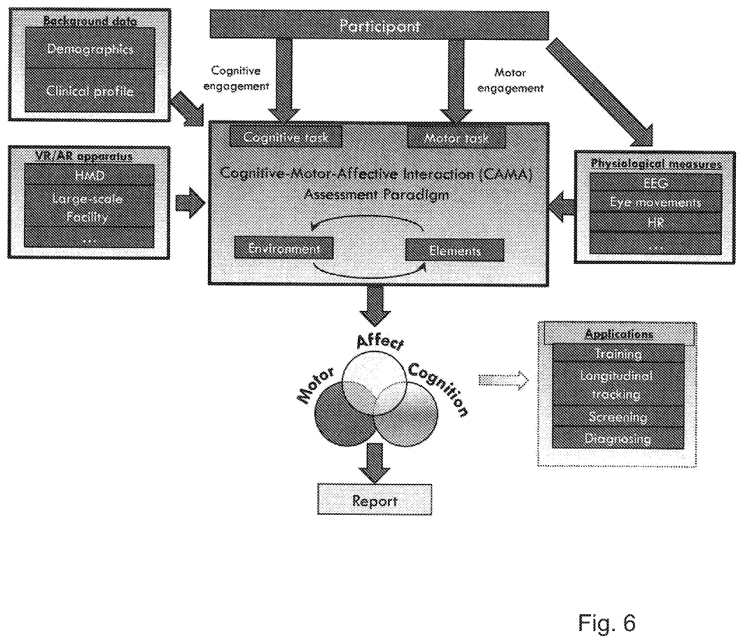

Errors were more prevalent during performance of the HMD-CTT (FIG. 6, p≤0.004). Interestingly, a significant group effect was found only for HMD-CTT (Trails 404 A and B; H″8.8, p≤0.012) but not for the pencil-and-paper CTT (H≤2.96; p≤0.227). Post-hoc analyses revealed that the significant Group effect for HMD-CTT was attributable to more errors among the OLD than the YA (p=0.009).

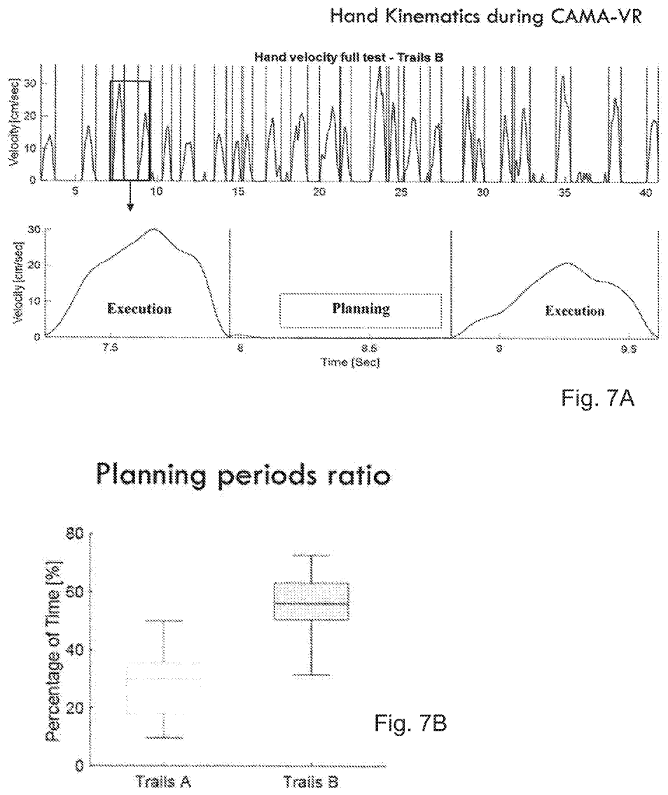

Correlations between pencil-and-paper and HMD-CTT—completion times FIG. 7 shows the relationship between performance on the pencil-and-paper and HMD-CTT for the YA (blue), MA (orange) and OLD (green) group. The Spearman correlation (rho; rₛ) between Part A completion time (t_{A}) on the gold-standard pencil-and-paper CTT and the corresponding Part A completion time on the HMD-CTT was 0.62 (p<0.001;. For Part B completion time (t_{B}), the Spearman correlation was 0.69 (p<0.001;).

Qualitative analysis of manual performance FIG. 8 shows grand averages of the scaled ball-to-ball hand trajectory velocity profiles for all participants who completed HMD-CTT Trails A (solid traces) and B (dashed traces). Data for the three age groups is color-coded (see legend for details). Based on these traces, the following observations can be made:
(1) For all groups and for both Trails A and B, movement toward a target (ball) does not stop immediately upon reaching the target (virtually touching, at x=100% which for all, except for the last trajectory, is also x=0% of the next trajectory), but little later, as reflected by the ensuing gradual decrease in velocity decrease at x=0%, reaching a minimum and ensuing gradual increase in velocity. This initial decrease on the grand average traces does not reach zero because the minimum is reached at a different time for each of the 24 individual ball-to-ball trajectories.
(2) For Trails A, but not for Trails B, soon after reaching this minimum (i.e., completing the execution of the previous trajectory), a new trajectory can be identified (time of emergence is designated by the left black arrow). The velocity profile of this trajectory is characterized by an accelerating portion (peak indicated by a gray arrow) and a decelerating portion upon approaching the target ball. The degree of asymmetry between these portions of the trajectory varies between groups, with YA showing greater symmetry.
(3) Conversely, for Trails B, a prolonged ‘executive function’ period is evident, and acceleration toward the target is identifiable after at least 40% of the trace, with the older groups showing a more delayed initiation of movement (black arrows on dashed traces). This pattern is consistent with the divided attention aspect of Trails B, in which the participant must locate the ball next in the numerical sequence but of opposite color to the current ball, ignoring the distracter ball with the correct number but the incorrect color (i.e., same color as the current ball).
(4) Consistent with the results for completion times (t_{A}, t_{B}), the velocity profiles for Trails A are faster than those of Trails B.

Test-retest reliability (Studies 1 & 2) For the DOME-CTT (retest interval of about 12 weeks), moderate reliability was found: Trails A (tA) and B (tB) (ICC=0.597, p=0.023, ICC=0.676, p=0.018, respectively). For the pencil-and-paper CTT, comparatively better reliability was found for Trails A (tA) (ICC=0.778, p=0.001) and Trails B (tB) (ICC=0.622, p=0.040). For the HMD-CTT (retest interval of about 12 weeks), moderate reliability was found for Trails A (tA) and B (tB) (ICC=0.618, p=0.004; ICC=0.593, p=0.003, respectively). For the pencil-and-paper CTT, comparatively better reliability was found for Trails A (tA) and Trails B (tB) (ICC=0.744, p<0.001, ICC=0.769, p<0.001, respectively).

In both VR-CTT formats, there were no substantial differences in ICC values for MA participants who engaged in cognitive training during the 12 week period (as part of the larger study protocol) as compared with those who did not. For the HMD-CTT (retest interval of about 2 weeks), good reliability was found for Trails A (tA) and B (tB) (ICC=0.748, p=0.002; ICC=0.893, p<0.001, respectively). The paper-and-pencil CTT also showed good reliability for both Trails A (tA) and Trails B (tB); Compared to HMD-CTT, the gold standard test had higher reliability for tA and 466 lower reliability for tB (ICC=0.851, p<0.001; ICC=0.798, p=0.001, respectively).

### Discriminant Validity (Studies 1 & 2)

Table 3 shows AUC values from ROC curves. Pencil-and-paper CTT AUC values were compared with AUC values obtained for each of the VR adaptations (i.e., DOME-CTT, HMD-CTT). For each VR adaptation, the relative difference [%] in AUC from the pencil-and-paper AUC is shown. Comparisons were made separately for Trails A and Trails B.

The data indicate that all CTT versions have relatively high discriminant validity (AUC″ 0.70; p≤0.05). AUCs are largely comparable, though slightly reduced for the VR adaptations.

### Comparing Completion Times Between DOME-CTT and HMD-CTT

The completion times suggest that tA and tB are higher (i.e., longer) for DOME-CTT relative to HMD-CTT. None of the participants completed both DOME-CTT and HMD-CTT testing, and the present work was not designed to compare between the two VR platforms. However, Applicant conducted analyses to address this question based on the existing evidence. The methodology and the results suggest shorter completion times for HMD-CTT as compared to the DOME-CTT (Tables 1 and 2).

### DISCUSSION

In this report, Applicant describes the development and validation of VR-based Color Trails Test (CTT), a traditional test of attention and processing speed, using two different types of VR systems. The first VR-based system involves projection of visual stimuli on the walls of a large, dome-shaped room (akin to a cave, monoscopic projection). The second VR-based system is a low-cost head-mount VR device (HMD) worn by the participant and suitable for home-based assessment. Adherence and usability of both VR-based versions proved to be relatively good, with only two participants (˜1.5%) not completing the VR tasks. Participants only rarely complained about the difficulty of completing the VR tasks, though there were no such complaints for the pencil-and-paper version. Our discussion integrates the results from two studies, each of which evaluated one of the VR-based versions.

### Construct Validity

Our results suggest that the new VR-based versions and gold standard pencil-and-paper version share similar psychometric properties (e.g., longer completion time for B vs. A). Coupled with the relatively high correlations between corresponding parts (˜0.7), this suggests that the VR and the pencil-and-paper tests measure the same cognitive constructs (e.g., sustained, divided attention). By comparison, in a cross-validation study of the TMT and CTT, Dugbartey and colleagues reported lower correlation values of 0.35 for Trails A and 0.45 for Trails B.

Notably, construct validity correlations for YA participants on the Trails A portion of the test were not significant. Applicant attributes this result to a ceiling effect in that to values by the VR task as compared to one page distribution of the targets in the pencil-and-paper CTT), greater task difficulty and/or the cognitive-motor interactions relevant to the VR versions but not the original pencil-and-paper test. Perceptual factors must also be considered. One possible account for this finding relates to different levels of visual immersion between the tests. The HMD-CTT provides no visual feedback from the arms, and the participant's subjective experience consists solely of moving the avatar (red ball) within the VR environment. In contrast, during the DOME-CTT, the participant sees his/her hand holding the wand-like stick in addition to the virtual avatar as s/he makes reaching movements toward the target balls. The latter configuration may complicate sensorimotor integration given the two parallel, relevant sensory input streams (physical hand, virtual avatar). A potential contributor to this complication is that participants can see their arms in full stereoscopic vision but the balls only in monoscopic projection. Subramanian and Levin reported superior motor performance (reaching movements) among healthy adults in a large-scale screen-based VR system as compared to an HMD-based system, apparently at odds with the present findings (i.e., slower movements for DOME-CTT as compared with HMD-CTT). Likely technical-methodological differences between the studies account for the disparity. For example, the HMD field of view was smaller (˜100° vs. ˜50° in the Subramanian and Levin study, and the type of task (reaching vs. consecutive trails making following rules) was markedly different.

### Cognitive-Motor Interactions

Our qualitative data clearly demonstrate that when shifting from a cognitive task primarily involving sustained attention (Trails A) to one that primarily involving divided attention (Trails B), upper-limb motor behavior changes. Previous research has employed VR to evaluate cognitive-motor interactions mainly in the context of locomotion. Most of the studies have reported clinical benefits related to cognitive-motor interactions associated with immersion in a VR environment.

In the current study, Applicant began exploring the effect of divided attention (operationalized as HMD-CTT Trails B performance) on the planning and execution of upper-limb reaching movements. The well-documented single-peak velocity profile typical of ballistic movements appears to govern the hand trajectories generated during HMD-CTT Trails A, a sustained attention task, but not during Trails B. In Trails B, a divided attention task, trajectories are characterized by an initial slow increase in the velocity profile, probably reflecting neural processes more related to executive function and less to motor execution. Potential age effects (e.g., less symmetric peak, slower overall velocity) are apparent in comparing the velocity profile across age groups.

Follow up studies will focus on developing reliable quantification methods and metrics to assess these cognitive-motor interaction effects. Notably, comparing the velocity profiles generated during a three-dimensional (3D) VR-based task to a classical two-dimensional (2D) task as the ‘gold standard’ is suboptimal, mainly due to the absence of a reliable theoretical model for three-dimensional hand-reaching movements. Thus, new referencing methodologies like sampling single target-to-target trajectories should be included as part of future versions and analyses of VR-based CTT tasks like those used here.

### Discriminant Validity

Further, both the traditional and VR-based versions demonstrated relatively high discriminant validity as reflected by high AUC values. These observations are consistent with the strong correlations for completion times between the VR-based and original CTT for each age group (with the exception of to in YA) as well as when combining participants across age groups.

Comparability in discriminant validity between VR-based and the gold standard CTT for completion times is encouraging. However, VR-based testing affords additional metrics that may be better at differentiating among age groups as well as between healthy and cognitively impaired individuals. Indeed, VR facilitates the development of new parameters of greater relevance to daily living (i.e., more ecologically valid) in that they better capture complex, integrated behaviors. Thus, Applicant believes that using such VR-based parameterization of multimodal function (e.g., hand-gaze coordination combined with hand trajectories) will provide superior discriminant validity.

### Test Retest Reliability

For a retest period of ˜12 weeks, the VR-based CTT adaptations showed moderate reliability (intraclass correlation of ˜0.6), while the pencil-and-paper version showed generally better reliability. The superior reliability of the original CTT for this retest interval may be attributable to the greater familiarity of the pencil-and-paper format, which may have led to a larger learning effect upon retest and consequently poorer reliability for the VR-based versions. Applicant also acknowledges that some middle-aged participants had engaged in a cognitive training protocol during the 12-week interval which may compromise test-retest evaluations.

However, for a retest period of ˜2 weeks, both the HMD-CTT and the original CTT showed good reliability (intraclass correlation of !0.75), with the VR-based adaptation showing superior reliability for Trails B. Our results are consistent with findings that shorter retest intervals yield higher reliability coefficients). As there does not appear to be a clear convention for the ideal test-retest interval), our data reporting reasonable reliability for both intervals is relevant and informative. Still, given that our sample sizes were small, the findings should be replicated in larger studies.

VR technologies enable us to enrich the current VR-based versions of the CTT to further enhance ecological relevance, mainly in the sense of engaging more modalities, and inter-modalities interactions. The challenge will then be how to leverage multimodal measures to understand such real-world processes as cognitive-motor interference during multi-tasking and ultimately assess function in a predictive or clinically meaningful way.

### CONCLUSIONS

In sum, the present study describes the development and validation of large-scale DOME-CTT) and head-mount (HMD-CTT) VR adaptations of the classic pencil-and-paper Color Trails Test (CTT) and provides key validation data, including construct validity relative to the original test, discriminant validity among age groups, and test-retest reliability at two different retest intervals. Critically, this work demonstrates the feasibility and viability of converting a neuropsychological test from two-dimensional pencil-and-paper to three-dimensional VR based format while preserving core features of the task and assessing the same cognitive functions. Our novel findings on the relationship between classical cognitive performance and upper-limb motor planning and execution lead to new analysis methods for other more ecological VR-based neuropsychological tests that incorporate cognitive-motor interactions.

List of abbreviations ANOVA analysis of variance; AUC—area under the curve; CTT-color trails test; FOV field of view; HMD head mounted device; ICC—intraclass correlation coefficients; MA middle-aged adults; MET multiple errands test; ROC—receiver operating characteristic; SD standard deviation; TMT—trail making test; VET virtual errands test; VR virtual reality; YA—young adults.

As a result of having demonstrated the feasibility of and viability of a neuropsychological test using a three-dimensional VR based format, Applicant has gone well beyond that and has developed an Extended Reality assessment method and system that integrates multiple domains from among cognitive, motor, ANS (emotion & affect, etc.) and sensory.

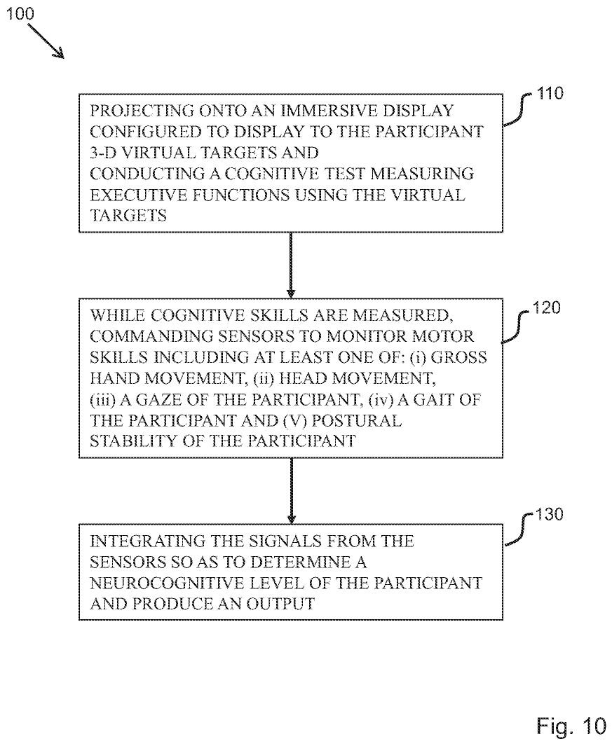

In one embodiment, the flow chart of FIG. 10 shows a computer-implemented method 100 of implementing an extended reality (XR) neuropsychological (or neurocognitive) test of a person using software or programmable instructions (hereinafter “software”) stored on a non-transitory computer-readable medium (as part of the overall memory of a computer system), the software executed by a processor (or processors) of the computer system, the software performing the following steps of method 100. Method 100 comprises a step 110 of projecting onto an immersive display (which may be an eye-mounted or head-mounted or large scale display) configured to display to the person three-dimensionally a series of virtual objects and conducting a first cognitive test that measures cognitive skills, including executive functions, using the series of virtual objects. The virtual objects may be virtual targets and may be either stationary or are dynamic, i.e. perceived by the person as moving (or flowing), for example at different rates and/or in different directions. The software may conduct the test by interfacing with and prompting the person with visual or other cues to take actions relating to the virtual objects as part of the cognitive test (one non-limiting example being moving an input device from target to target).

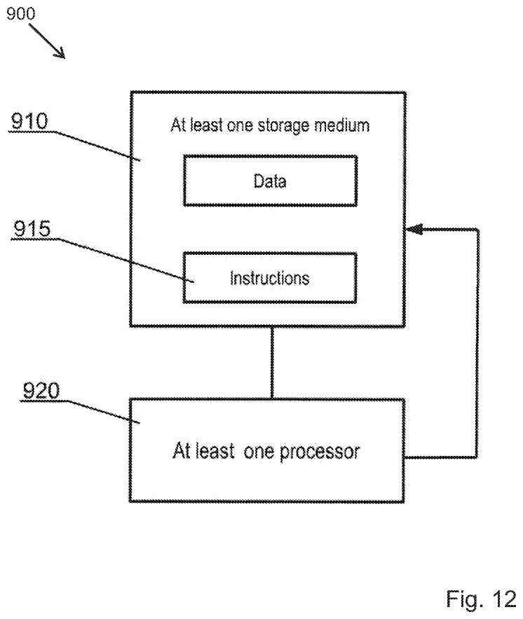

The processor(s) is part of the computer system 900 (FIG. 12). A memory storage component 910 of the computer system, is configured to receive and store the inputs. In general, the computer system, in all embodiments, includes all necessary hardware and software 915 to perform the functions described herein.

The executive functions include, in some embodiments, at least one of planning, sustained visual attention (SVA), divided attention (DA), verbal memory (VM), spatial orientation and task switching, sequencing. They may also include a correlation between any two executive functions. In some versions, the executive functions include two of or at least two of (or three of or at least of) or all of (or any combination in between): planning, sustained visual attention (SVA), divided attention (DA), episodic memory, spatial orientation and task switching, sequencing, mental flexibility, visual scanning, information processing, problem solving, abstraction, impulse control.

One implementation of Extended Reality is Virtual Reality, but there are other computer-altered realities namely Augmented Reality (AR), Mixed Reality (MR).

In step 120, while measuring the cognitive skills involving the executive functions, the software may perform a step of commanding sensors to monitor or measure motor skills (or measured), including at least one of: (i) gross hand movement by commanding a motion sensor, (ii) head movement by commanding a motion sensor, (iii) an eye movement (one example being a gaze) of the person at the virtual objects by commanding eye tracking, (iv) a gait of the person by commanding at least one mobility sensor (the mobility sensors may include one or more inertial movement units (IMU)), motion capture system sensors (for example reflective markers that may be filmed with a camera or an image system), force plate sensors or insole sensors and (v) postural stability of the person by commanding at least one of mobility sensors (IMUs), force plate sensors and insole sensors.

In one non-limiting embodiment, the motion capture system sensors include reflective markers situated on a body of the person (for example on a foot or on each foot or on another part of the body). In some versions, the motor skills are monitored while the person is walking on a treadmill using self-paced walking, fixed paced walking, or walking overground while immersed in the extended reality environment (which may be a virtual environment).

In some cases, the gross hand movement involves reaching movements by the person to hit the virtual objects.

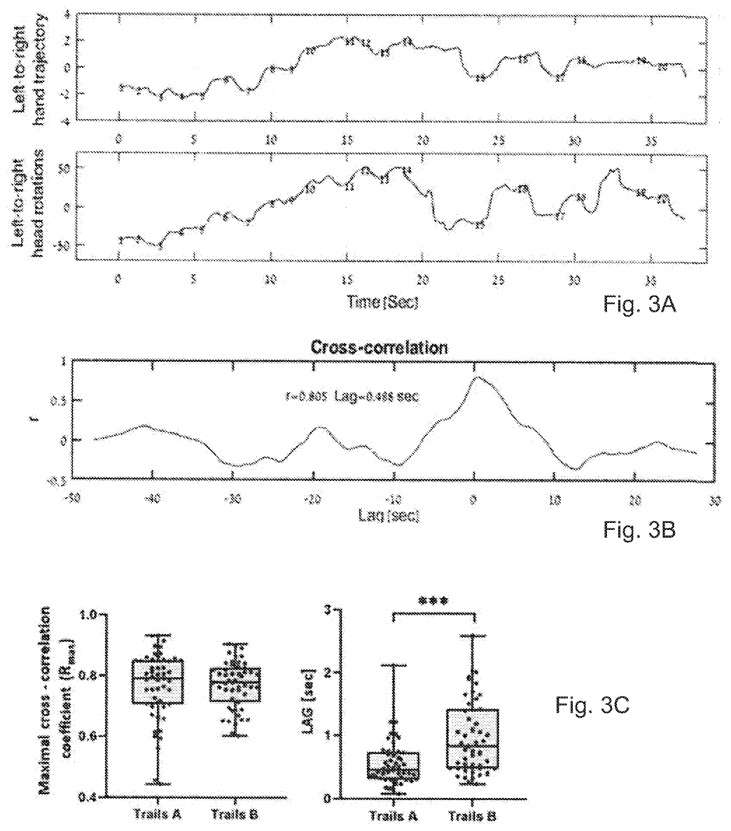

The eye tracking as well as a pupil dilation may also be recorded to assess cognitive processing and visual exploration patterns. In some cases, an invisible unity object representing a gaze position of the person at any given moment records three-dimensional coordinates of the gaze along with three-dimensional coordinates of the hand and head of the person. This allows a comparison to be made, as shown for example in FIG. 3A, FIG. 3B, FIG. 3C. For example, method step 120 may further comprise comparing a waveform of the gross hand movement and a waveform of an eye movement of the person.

In another version, method 120 may further comprise comparing between hand movement and head movement of the same person. An example of comparing a waveform of the gross hand movement and a waveform of the head movement of the person to measure head-hand coordination as seen in FIG. 3A.

Applicant administrated the cross-correlation function on two vectors of data of hand horizontal trajectory profile and head rotation angels (both in 1D right-to-left direction, see cross-correlation graph of FIG. 3B) of the participants during the tests. Best-fit coefficient, and phase shift (i.e., time lag) between the signals were registered. Furthermore, Applicant recorded the total completion time of each participant (as in the standard case of the CTT test) in both trails. Paired, two-tailed t tests were conducted to detect potential statistically significant differences in lag time of hand motion and head rotation and in total completion time in trails A versus trails B. The statistical significance level was set as p<0.05.

Examples of the horizontal hand trajectory profile and head rotation angles (FIG. 3A) as well as the cross-correlation analysis (see cross-correlation graph of FIG. 3B) are calculated by our dedicated code, for one participant who performed Trail A.

The level of spatial coherence between the hand and the head motions was found to be similarly high in both Trails A and B, as demonstrated from the resulted values of the coefficient of best-fit as follows, 0.76±0.11 (mean±SD) and 0.77.6±0.07 for Trails A and Trails B, respectively (FIG. 3C). The above findings indicate a stable spatial coordination between the motions throughout the trails.

The phase shift between the hand and the head motions, extracted from the best-fit configuration of the superimposed signals, revealed alterations in temporal synchronization when participant performed Trails B. A significant increase in time lags in which the head leads the hand pattern was documented, with average lags of 0.54±0.35 seconds and 0.96±0.57 seconds for Trails A and Trails B, respectively (see LAG graph on the far right of FIG. 3C).

In some embodiments, step 120 further comprises sensing motor skill kinematics (one example of which is hand kinematics or kinematics of a head or other body portion) of the person and dividing, by the processor(s), a motor skill kinematics waveform (such as a hand kinematic waveform) into at least one planning portion and at least one execution portion while monitoring a velocity of a hand of the person to ascertain a planning period ratio that correlates with the person's processing speed and motor execution speed. (See, for example, measurements indicated in FIGS. 7A and 7B.) In some versions, it further comprises extracting a level of hand movements during the planning periods to obtain indications of a level of impulsivity versus caution of the person. It may also include monitoring a trajectory smoothness of the hand of the person. This refers to the amount of involuntary shaking or movement of the hand as the hand moves along a trajectory, for example from one virtual object to another.

The Extended Reality cognitive-affective-motor assessment method may utilize an input device on the body of the person or held by the person in their hand. The input device may be activated by hand movement or by head movement (in the case of a head-mounted input device). The monitoring of the motor skills may include monitoring the head movement, the gross hand movement and the gait of the person. In another example, the monitoring of the motor skills includes monitoring the gait of the person while the person is doing at least one of walking, running and standing.

Step 130 of method 100 may comprise integrating the signals from the sensors so as to determine a neurocognitive level or a motor skills level (and in some cases both a neurocognitive level and a motor skills level) of the person. In some embodiments, this step may involve integrating the signals so as to determine the relation between motors skills levels and neurocognitive level (and in some cases both) on affective state. Examples of the implementation of this “relation” were stated above in terms of a person's head rotation (a motor skill) and the person's hand movement (a motor skill) being both tracked while monitoring a cognitive skill, e.g., sustained visual attention (SVA), and the ratio (which is a type of “correlation coefficient”) between them. This step 130 may also include producing an output reflecting the determined neurocognitive level (and motor level in some cases), such as by a score.

The neurocognitive level may be determined by taking into account the effect of the motor skills performance on the cognitive tests. The motor skills level may be determined by taking into consideration the effect of the cognitive skills performance on the person's performance on the motor skills monitored.

Step 130 may comprise determining a score for the output including by contrasting the person's performance under a first condition with the person's performance under a second condition that differs from the first condition with respect to one or more test parameters. One non-limiting example of such a condition would be whether or not the virtual objects are numbered or not numbered.

The score or neurocognitive level may be calculated in a number of ways. For example, the software may execute a step in which the neurocognitive level is calculated based on a ratio of an initial score or a tentative score reflecting the person's performance on the cognitive test (i) to a baseline score of that person on that test or (ii) to a baseline score of the population (or a segment of the population resembling that person). Alternatively, the ratio may be between the initial or tentative score and a predetermined threshold score that is considered to be of significance. The same method of calculating applies to determining a score on the motor skills level. The same method of calculating applies as to determining affective states.

In some cases, in other to make a comparison of the person's performance on one test to their performance on the same test with altered conditions, in order to learn the effect of stress on that person, the method may further comprise applying to the person unexpected or challenging movements to elevate a level of difficulty of the motor skills such as by generating physical perturbations that induce stress during the monitoring of the motor skills and/or the testing of the cognitive skills. The perturbations may be periodic or non-periodic. For example, the postural stability of the person may be perturbed during, before, or after measuring the cognitive skills.

In addition to the cognitive and motor domains, the person's emotion & affect (sometimes referred to as “ANS” or simply “affect”) may also be monitored and sensory signals may be applied to influence the person's performance on the cognitive, motor and/or affect testing/monitoring/measuring. In that case, method 100 may further comprising at least one of (a) sensing an affect of the person by sensing at least one of galvanic skin response (GSR), ECG, respiration and skin temperature; and (b) applying sensory signals in an XR environment (in one example to manipulate mental stress levels), including by using at least one of (i) visual colors or shapes or other scenery within the scene viewed by the person on the display during the cognitive test, (ii) sounds heard by the person during the test, and (iii) tactile sensations felt by the person such as by use of a haptic glove on one or both hands of the person to affect an outcome of the cognitive testing, motor skills monitoring and/or affect sensing of the person. In certain embodiments, the sensory signals may comprise olfactory sensations induced by pleasant or unpleasant odor-producing agents such as sprays.

In the case when affective has been monitored, step 130 of method 100 may comprise integrating the signals from the sensors so as to determine a score of the relation between motor skills level and neurocognitive level (and in some cases both) on affective state. In addition, the motor skills level and the neurocognitive level (or both) may be determined by taking into consideration affective conditions (e.g., stress, relaxation) that are monitored by the sensors (ECG, respiration, GSR etc.). The affective conditions may also be determined by taking into account the performance of motor skills performance, or the neurocognitive performance (or both) as monitored.

Furthermore, instead of measuring motor skills while measuring cognitive skills, the method 100 may involve instead measuring any other combinations of two (or three) of the cognitive, motor and affect domains depicted in FIG. 1.

Accordingly, in some embodiments, step 130 of method 100 further comprises determining, by the processor, the neurocognitive and motor level of the person based at least in part on at least one of (or at least two or at least three of or at least four of or all of) the following:
(a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching;
(b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching;
(c) changing a level of physical difficulty of motor skills by applying or changing physical perturbation as part of the measurement of a particular domain (such as cognitive or motor) in response to detection of a particular affective state. A practical illustration of this is where the person's performance on affective tests indicates a significant amount (for example in relation to a predetermined numerical threshold) of anxiety. If the person is being tested for a desk job not requiring being able to handle a lot of stress or the person already works in such a job, since measurement of anxiety is not needed, one would then reduce the stress perturbation level.
(d) comparing the motor skills of the person utilizing only eye movement by the person, with motor skills of the person using only head movement by the person, or comparing motor skills using one hand of the person only versus motor skills using both hands of the person; and
(e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

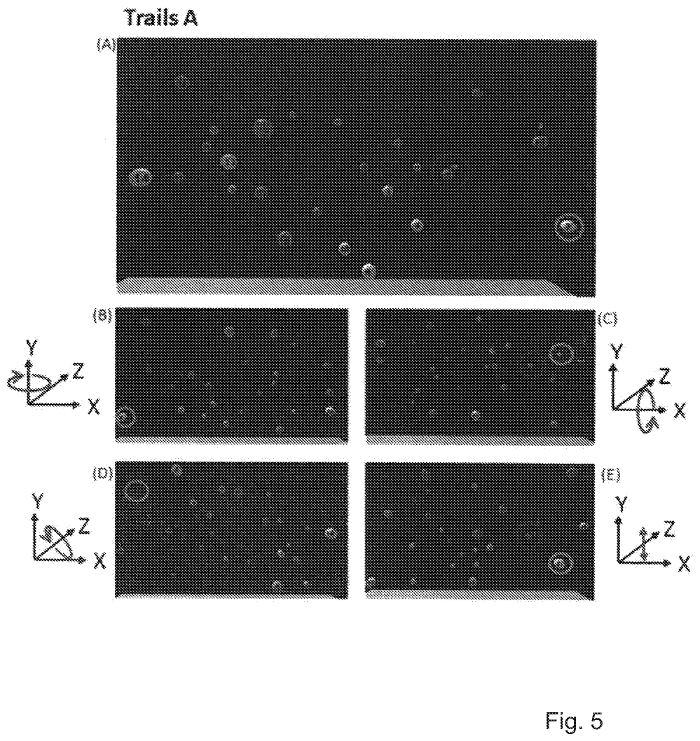

As shown in FIG. 5, one advantage of the method or system of the XR Assessment described herein, in accordance with certain embodiments, is that many different configurations can be generated. This is particularly helpful because if the person being tested repeats a configuration, that person recalls some of the positions of the virtual objects and this affects their performance on the test. In some embodiments, an unlimited number or a virtually unlimited or many thousands, of configured can be generated by rotating the scene viewable by the person in the extended reality environment, as well as by flipping (mirroring) the scene by generating a mirror image thereof.

Accordingly, in certain embodiments of method 100 further comprises generating a multiplicity of or a series of or dozens of or hundreds of or thousands (or a virtually unlimited number) of configurations of the extended reality (ER) neuropsychological tests of the person by conducting the following steps:
(1) situating the display screen on the person during the first cognitive test and during a second cognitive test in a first configuration, where the second cognitive test is configured to display the series of virtual objects so as to distract an attention of the person more than the first cognitive test;
(2) wherein a number, axis, angle and order of rotations and spatial flips are randomized, rotating a scene viewable on the display screen by the person along at least one of the X, Y and Z axes and in some cases also spatially flipping the scene, to create a new configuration such that (i) a total distance of a trail from the first virtual object to the last virtual object of a particular cognitive test is unchanged from one configuration to another configuration and such that (ii) the distance and trajectory between any one of the virtual objects and a next numbered virtual object are identical between the first cognitive test of a particular configuration and the second cognitive test of the particular configuration.

The rotating can be at any angle—therefore an unlimited number of possibilities/configurations can be created. Accordingly, a further substep of method 100 is generating a multiplicity of or dozens or hundreds or thousands of configurations of the extended reality neuropsychological tests of the person by varying an angle of the rotations of the scene.

In any embodiment, step 130 of the method 100 may further comprise scoring the person's performance on a cognitive level based on a completion time of the test or based on a single or a range of a numeric or alphabetic indicia.

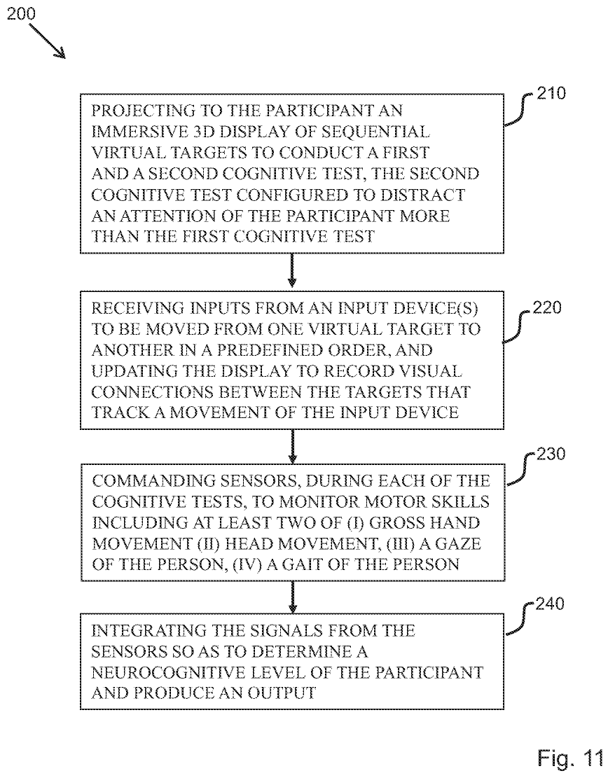

As shown in the flow chart of FIG. 11, another embodiment is a computer-implemented method 200 of implementing extended reality (XR) neuropsychological tests of a person using software or other programmable instructions (hereinafter “software”). The software is stored on a non-transitory computer-readable medium (as part of the overall memory of the computer system) and is executed by a processor(s) of the computer system so as to perform the following steps: Method 200 may comprise a first step 210 of projecting onto an immersive XR display (for example an eye-mounted or head-mounted or large scale display configured to display to the person three-dimensionally a series of sequentially identified virtual targets so as to conduct a first cognitive test that measures executive functions and so as to conduct a second cognitive test that measures executive functions, the second cognitive test configured to display the series of virtual targets so as to distract an attention of the person more than the first cognitive test. The virtual targets may be virtual objects that are either stationary or are dynamic, i.e. perceived by the person as moving (or flowing), for example at different rates and/or in different directions. The software may conduct the test by interfacing with and prompting the person with visual or other cues to take actions relating to the virtual targets as part of the cognitive test (one non-limiting example being moving an input device from target to target).

The processor(s) is part of a computer system 900 (FIG. 12). A memory storage component 910 of the computer system, is configured to receive and store the inputs. In general, the computer system, in all embodiments, includes all necessary hardware and software 915 to perform the functions described herein.

Step 220 may comprise receiving input from at least one of (i) a handheld and (ii) head-mounted input device moved by the person from one virtual target to another until a predefined number of the virtual targets have been contacted in a predefined order, and instructing the XR display to update so as to record visual connections between the targets that track a movement of the handheld input device, when used, and of the head-mounted input device, when used. If the handheld input device and the head-mounted input device are both used, they can be used simultaneously or non-simultaneously.

A third step 230 may comprise commanding sensors, during each of the first cognitive test and the second cognitive test to monitor motor skills of the person including at least two of (i) gross hand movement by instructing a motion sensor, (ii) head movement by instructing a motion sensor, (iii) an eye movement (for example a gaze) of the person at the virtual targets by instructing an eye tracking sensor, (iv) a gait of the person by instructing at least one of mobility sensors, motion capture system sensors, force plate sensors or insole sensors and (v) postural stability of the person by instructing at least one of mobility sensors, force plate sensors and insole sensors.

Step 240 may include integrating the signals from the sensors so as to determine a neurocognitive level or a motor skills level (and in some cases both a neurocognitive and a motor skills level) of the person and produce an output reflecting the determined level, such as a score. The neurocognitive level may be determined by taking into account the effect of the motor skills performance on the cognitive tests. The motor skills level may be determined by taking into consideration the effect of the cognitive skills performance on the person's performance on the motor skills monitored.

In some embodiments, step 240 may integrate the signals from the sensors so as to determine a neurocognitive level or a motor skills level (and in some cases both a neurocognitive and a motor skills level), and affective states (conditions) of the person and produce an output reflecting the determined level, such as a score. The neurocognitive level may be determined by taking into account the effect of the motor skills performance and the affective conditions on the cognitive tests. The motor skills level may be determined by taking into consideration the effect of the cognitive skills performance and the affective conditions on the person's performance on the motor skills monitored. The affective conditions may be determined by taking into account the performance of motor skills performance, or the neurocognitive performance (or both) as measured/monitored.

Step 240 may comprise determining a score for the output including by contrasting the person's performance under a first condition with the person's performance under a second condition that differs from the first condition with respect to one or more test parameters. One non-limiting example of such a condition would be whether or not the virtual targets are numbered or not numbered. The discussion of the possible ways of integrating the signals and determining the score as discussed with respect to the method step 130 of method 100 applies equally to the method 200.

Each of the variations previously described herein with respect to method 100 apply equally to method 200. This includes, for example, the details and variations about the monitoring of the motor skills (eye tracking and head movement in one version, head movement, the gross hand movement and the gait of the person in another version and the gait of the person while the person is doing at least one of walking, running and standing in another version), the motion capture system sensors may be reflective markers situated on each foot of the person and the motor skills are monitored while the person is walking on a treadmill using self-paced walking, fixed paced walking, or walking overground while immersed in the virtual environment, applying movements such as sudden movement that increase a level of difficulty of the motor skill or by adding physical perturbations to generate physical stress, at least one of (a) sensing an affect of the person by sensing at least one of galvanic skin response (GSR), ECG, respiration and skin temperature; and (b) applying sensory signals in an XR environment in order to manipulate mental stress levels, including by using at least one of (i) colors or shapes, (ii) sounds and (iii) tactile sensations such as by use of a haptic glove on one or both hands of the person, to affect an outcome of the cognitive testing, motor skills monitoring and/or affect sensing of the person, the types of the executive functions (for example sustained visual attention (SVA), divided attention (DA), planning, episodic memory, spatial orientation and task switching, sequencing, mental flexibility, visual scanning, information processing, problem solving, abstraction, impulse control. In certain embodiments, the sensory signals may comprise olfactory sensations induced by pleasant or unpleasant odor-producing agents such as sprays.

In the case when affective has been monitored, step 240 of method 200 may comprise integrating the signals from the sensors so as to determine a score of the relation between motor skills level and neurocognitive level (and in some cases both) on affective state. In addition, the motor skills level and the neurocognitive level (or both) may be determined by taking into consideration affective conditions (e.g., stress, relaxation) that are monitored by the sensors (ECG, respiration, GSR etc.). The affective conditions may also be determined by taking into account the performance of motor skills performance, or the neurocognitive performance (or both) as monitored.

Method 200 also may comprise sensing motor skill kinematics (for example hand kinematics) of the person and dividing, by the processor, a motor skill kinematic waveform into at least one planning portion and at least one execution portion with all the details described regarding method 100, the gross hand movement may involve reaching movements by the person to hit the virtual targets, the eye tracking and a pupil dilation may be recorded to assess cognitive processing and visual exploration patterns, and wherein an invisible unity object representing a gaze position at any given moment records three-dimensional coordinates of the gaze along with three-dimensional coordinates of the hand and head of the person.

In some embodiments, method 200 may further comprise comparing a waveform of the gross hand movement and a waveform of an eye movement of the person or comparing a waveform of the gross hand movement and a waveform of the head movement of the person to measure head-hand coordination.

Method 200, in some embodiments, likewise may further comprise determining, by the processor, the neurocognitive and motor level of the person based at least in part on at least one of or on at least two of:
(a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching;
(b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching;
(c) changing a level of difficulty of motor skills by for example introducing physical perturbation or sudden movement (for example movement of a treadmill suddenly) in response to detection of a particular affective state;
(d) whether the motor skills were monitored utilizing eye movement by the person only, head movement by the person only, one hand of the person only or both hands of the person only; and
(e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

In addition, method 200 may further comprise generating a multiplicity of or a series of such as dozens of or hundreds of or thousands of spatially distinct configurations of the extended reality neuropsychological tests of the person using the rotating or rotating and flipping described with respect to method 100 (with all possible variations). As in method 100, in method 200, the scoring of the person's performance on a cognitive level may be based on a completion time of the test or based on a single or a range of a numeric or alphabetic indicia.

FIG. 12 is a schematic illustration of a computer-implemented system 900, in accordance with one embodiment of the invention, for implementing methods 100, 200. The system 900 may include at least one non-transitory computer-readable or processor-readable storage medium 910 that stores at least one of processor-executable instructions 915 or data; and at least one processor 920 communicably coupled to the at least one non-transitory processor-readable storage medium 910. The at least one processor 920 may be configured to (by executing the software or instructions 915) perform the steps of method 100 and of method 200 in any variation thereof.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A computer-implemented method of implementing an extended reality (XR) neuropsychological test of a person using software stored on a non-transitory computer-readable medium, the software executed by a processor, the software performing the following:
projecting onto an immersive display, that is configured to display to the person three-dimensionally, a series of virtual objects and conducting a first cognitive test that measures cognitive skills, including executive functions, using the series of virtual objects;
while measuring the cognitive skills involving the executive functions, commanding sensors to monitor motor skills of the person including at least one of: (i) gross hand movement by commanding a motion sensor, (ii) head movement by commanding a motion sensor, (iii) an eye movement at the virtual objects by commanding an eye tracking sensor, (iv) a gait of the person by commanding at least one of mobility sensors, motion capture system sensors, force plate sensors or insole sensors and (v) postural stability of the person by commanding at least one of mobility sensors, force plate sensors and insole sensors, and
integrating the signals from the sensors so as to determine a neurocognitive level or a motor skills level of the person and produce an output reflecting the determined level.

2. The method of claim 1, wherein the monitoring of the motor skills includes the eye tracking and monitoring the head movement,

3. The method of claim 1, wherein the monitoring of the motor skills includes monitoring the head movement, the gross hand movement and the gait of the person.

4. The method of claim 1, wherein the monitoring of the motor skills includes monitoring the gait of the person while the person is doing at least one of walking, running and standing.

5. The method of claim 1, further comprising applying to the person movement related perturbations to generate physical stress or increased difficulty during the monitoring of the motor skills and/or the testing of the cognitive skills.

6. The method of claim 1, further comprising at least one of
(a) sensing an affect of the person by sensing at least one of galvanic skin response (GSR), ECG, respiration and skin temperature; and
(b) applying sensory signals in an XR environment including by using at least one of (i) colors or shapes, (ii) sounds, and (iii) tactile sensations to affect an outcome of the cognitive testing, motor skills monitoring and/or affect sensing of the person.

7. The method of claim 1, wherein the executive functions include at least one of planning, sustained visual attention (SVA), divided attention (DA), spatial orientation and task switching, sequencing or a correlation between any two executive functions.

8. The method of claim 1, wherein the executive functions include at least two of planning, sustained visual attention (SVA), divided attention (DA), episodic memory, spatial orientation and task switching, sequencing, mental flexibility, visual scanning, information processing, problem solving, abstraction, impulse control.

9. The method of claim 1, further comprising sensing motor skill kinematics of the person and dividing, by the processor, a motor skill kinematic waveform into at least one planning portion and at least one execution portion while monitoring a velocity of a body portion of the person to ascertain a planning period ratio that correlates with the person's processing speed and motor execution speed.

10. The method of claim 9, further comprising extracting a level of hand movements during the planning periods to obtain indications of a level of impulsivity versus caution of the person.

11. The method of claim 1, wherein the gross hand movement involves reaching movements by the person to hit the virtual objects.

12. The method of claim 1, wherein the eye tracking and a pupil dilation are recorded to assess cognitive processing and visual exploration patterns, and wherein an invisible unity object representing a gaze position at any given moment records three-dimensional coordinates of the gaze along with three-dimensional coordinates of the hand and head of the person.

13. The method of claim 1, further comprising comparing a waveform of the gross hand movement and a waveform of an eye movement of the person.

14. The method of claim 1, further comprising comparing a waveform of the gross hand movement and a waveform of the head movement of the person to measure head-hand coordination.

15. The method of claim 1, further comprising the software performing operating a moving platform or treadmill configured for the person to stand, walk or run on so as to introduce physical perturbations.

16. The method of claim 15, wherein the motion capture system sensors are reflective markers situated on a body of the person and wherein the motor skills are monitored while the person is walking on the treadmill using self-paced walking, fixed paced walking, or walking overground while immersed in the virtual environment or extended reality.

17. The method of claim 1, further comprising determining, by the processor, the neurocognitive and motor level of the person based at least in part on at least one of:
(a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching;
(b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching;
(c) changing a level of physical difficulty of motor skills or cognitive skills by applying or changing physical perturbations in response to detection of a particular affective state;
(d) comparing the motor skills of the person utilizing only eye movement by the person, with motor skills of the person using only head movement by the person, or comparing motor skills using one hand of the person only versus motor skills using both hands of the person; and
(e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

18. The method of claim 1, further comprising determining, by the processor, the neurocognitive and motor level of the person based at least in part on at least two of:
(a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching;
(b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching;
(c) changing a level of physical difficulty of motor skills by applying or changing physical perturbations in response to detection of a particular affective state;
(d) comparing the motor skills of the person utilizing only eye movement by the person, with motor skills of the person using only head movement by the person, or comparing motor skills using one hand of the person only versus motor skills using both hands of the person; and
(e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

19. The method of claim 1, further comprising generating a multiplicity of configurations of the extended reality neuropsychological tests of the person by:
projecting onto the immersive display to the person three-dimensionally during the first cognitive test and during a second cognitive test in a first configuration, the second cognitive test configured to display the series of virtual objects so as to distract an attention of the person more than the first cognitive test;
wherein a number, axis, angle and order of rotations and spatial flips are randomized, rotating a scene viewable on the display screen by the person along at least one of the X, Y and Z axes and spatially flipping the scene, to create a new configuration such that (i) a total distance of a trail from the first virtual object to the last virtual object of a particular cognitive test is unchanged from one configuration to another configuration and such that (ii) the distance and trajectory between any one of the virtual objects and a next numbered virtual object are identical between the first cognitive test of a particular configuration and the second cognitive test of the particular configuration.

20. The method of claim 19, further comprising generating thousands of configurations of the extended reality neuropsychological tests of the person by varying an angle of the rotations.

21. The method of claim 1, further comprising various scoring a performance of the person on the neurocognitive level based on a completion time of the test or based on a single or a range of numeric or alphabetic indicia.

22. The method of claim 1, further comprising determining a score for the output including by contrasting the person's performance under a first condition with the person's performance under a second condition that differs from the first condition with respect to one or more test parameters.

23. The method of claim 1, wherein the virtual objects are perceived by the person as moving in different directions and/or at different rates.

24. A method of implementing extended reality (XR) neuropsychological tests of a person using software stored on a non-transitory computer-readable medium, the software executed by a processor, the software performing the following:
projecting onto an immersive XR display, that is configured to display to the person three-dimensionally, a series of sequentially identified virtual targets so as to conduct a first cognitive test that measures executive functions and so as to conduct a second cognitive test that measures executive functions, the second cognitive test configured to display the series of virtual targets so as to distract an attention of the person more than the first cognitive test;
receiving input from at least one of (i) a handheld and (ii) head-mounted input device that is moved by the person from one virtual target to another until a predefined number of the virtual targets have been contacted in a predefined order, and instructing the display to update the XR display so as to record visual connections between the targets that track a movement of the handheld input device, when used, and of the head-mounted input device, when used;
commanding sensors, during each of the first cognitive test and the second cognitive test to monitor motor skills of the person including at least two of (i) gross hand movement by commanding a motion sensor, (ii) head movement by commanding a motion sensor, (iii) an eye movement of the person at the virtual targets by commanding an eye tracking sensor, (iv) a gait of the person by commanding at least one of mobility sensors, motion capture system sensors, force plate sensors or insole sensors and (v) postural stability of the person by commanding at least one of mobility sensors, force plate sensors and insole sensors; and
integrating the signals from the sensors so as to determine a neurocognitive level or a motor skills level of the person and produce and output reflecting the determined level.

25. The method of claim 24, wherein the monitoring of the motor skills includes the eye tracking and monitoring the head movement.

26. The method of claim 24, wherein the monitoring of the motor skills includes monitoring the head movement, the gross hand movement and the gait of the person.

27. The method of claim 24, wherein the monitoring of the motor skills includes monitoring the gait of the person while the person is doing at least one of walking, running and standing.

28. The method of claim 24, further comprising applying to the person movement related perturbations to generate physical stress or increased difficulty of performance during the monitoring of the motor skills and/or the testing of the cognitive skills.

29. The method of claim 24, further comprising at least one of
(a) sensing an affect of the person by sensing at least one of galvanic skin response (GSR), ECG, respiration and skin temperature; and
(b) applying sensory signals in an XR environment in order to manipulate mental stress levels, including by using at least one of (i) colors or shapes, (ii) sounds, (iii) tactile sensations to affect an outcome of the cognitive testing, motor skills monitoring and/or affect sensing of the person.

30. The method of claim 24, wherein the executive functions include either sustained visual attention (SVA) or divided attention (DA) and at least one of planning, spatial orientation and task switching, sequencing or a correlation between any two executive functions.

31. The method of claim 24, wherein the executive functions include either sustained visual attention (SVA) or divided attention (DA) and at least two of planning, episodic memory, spatial orientation and task switching, sequencing, mental flexibility, visual scanning, information processing, problem solving, abstraction, impulse control.

32. The method of claim 24, further comprising sensing hand kinematics of the person and dividing, by the processor, a hand kinematic waveform into at least one planning portion and at least one execution portion while monitoring a velocity of a hand of the person to ascertain a planning period ratio that correlates with the person's processing speed and motor execution speed.

33. The method of claim 24, further comprising extracting a level of hand movements during the planning periods to obtain indications of a level of impulsivity versus caution of the person.

34. The method of claim 24, wherein the gross hand movement involves reaching movements by the person to hit the virtual targets.

35. The method of claim 24, wherein the eye tracking and a pupil dilation are recorded to assess cognitive processing and visual exploration patterns, and wherein an invisible unity object representing a gaze position at any given moment records three-dimensional coordinates of the gaze along with three-dimensional coordinates of the hand and head of the person.

36. The method of claim 24, further comprising comparing a waveform of the gross hand movement and a waveform of an eye movement of the person.

37. The method of claim 24, further comprising comparing a waveform of the gross hand movement and a waveform of the head movement of the person to measure head-hand coordination.

38. The method of claim 24, further comprising the software performing operating a moving platform or treadmill configured for the person to stand, walk or run on so as to introduce physical perturbations.

39. The method of claim 38, wherein the motion capture system sensors are reflective markers situated on each foot of the person and wherein the motor skills are monitored while the person is walking on the treadmill using self-paced walking, fixed paced walking, or walking overground while immersed in the virtual environment.

40. The method of claim 24, further comprising determining, by the processor, the neurocognitive and motor level of the person based at least in part on at least one of:
(a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching;
(b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching;
(c) changing a level of difficulty of performance of motor skills by introducing physical perturbations in response to detection of a particular affective state;
(d) whether the motor skills were monitored utilizing eye movement by the person only, head movement by the person only, one hand of the person only or both hands of the person only; and
(e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

41. The method of claim 24, further comprising determining, by the processor, the neurocognitive and motor level of the person based at least in part on at least two of:
(a) comparing a first motor control that is measured while performing one of planning, sustained visual attention (SVA), divided attention (DA), verbal memory (VM), spatial orientation and task switching, with a second motor control measured while performing a different one of planning, sustained visual attention (SVA), divided attention (DA), verbal memory (VM), spatial orientation and task switching;
(b) comparing a first affect measured while performing one of planning, sustained visual attention (SVA), divided attention (DA), verbal memory (VM), spatial orientation and task switching, with a second affect measured while performing a different one of planning, sustained visual attention (SVA), divided attention (DA), verbal memory (VM), spatial orientation and task switching;
(c) changing a level of performance of motor skills by introducing physical perturbations in response to detection of a particular affective state;
(d) whether the motor skills were monitored utilizing eye movement by the person only, head movement by the person only, one hand of the person only or both hands of the person only; and
(e) comparing (i) the person's skills in a particular domain tested in isolation, the particular domain selected from the cognitive, motor, affective, sensory domain, the sensory domain influencing testing of the cognitive, motor and affective domains, with (ii) the person's skills in the particular domain when tested in combination with all remaining domains from amongst the cognitive, motor, affective domains, as influenced by the sensory domain.

42. The method of claim 24, further comprising generating thousands of spatially distinct configurations of the extended reality neuropsychological tests of the person by:
projecting onto the immersive display to the person three-dimensionally during the first cognitive test and during a second cognitive test in a first configuration, the second cognitive test configured to display the series of virtual targets so as to distract an attention of the person more than the first cognitive test;
wherein a number, axis, angle and order of rotations and spatial flips are randomized, rotating a scene viewable on the display screen by the person along at least one of the X, Y and Z axes and spatially flipping the scene, to create a new configuration such that (i) a total distance of a trail from the first virtual target to the last virtual target of a particular cognitive test is unchanged from one configuration to another configuration and such that (ii) the distance and trajectory between any one of the virtual targets and a next numbered virtual target are identical between the first cognitive test of a particular configuration and the second cognitive test of the particular configuration.

43. The method of claim 24, further comprising generating an unlimited number of configurations of the extended reality neuropsychological tests of the person by varying an angle of the rotations.

44. The method of claim 24, further comprising scoring a performance of the person on the neurocognitive level based on a completion time of the test or based on a single or range of a numeric or alphabetic scores.

45. The method of claim 24, wherein the virtual targets are dynamic in that they are perceived by the person as moving in different directions and/or at different rates.

46. The method of claim 24, further comprising determining a score for the output including by contrasting the person's performance under a first condition with the person's performance under a second condition that differs from the first condition with respect to one or more test parameters.
